(19) United States
(12) Patent Application Publication
(10) Pub. No.: US 2026/0117289 A1
(43) Pub. Date: Apr. 30, 2026

(54) SAMPLE HANDLING SYSTEMS, DEVICES, AND METHODS

(71) Applicant: 10x Genomics, Inc., Pleasanton, CA (US)

(72) Inventor: Hanyoup Kim, Foster City, CA (US)

(21) Appl. No.: 19/364,291

(22) Filed: Oct. 21, 2025

Related U.S. Application Data
(60) Provisional application No. 63/714,559, filed on Oct. 31, 2024.

(51) Int. Cl. C12Q 1/6841; B01L 7/00

(52) U.S. Cl. CPC C12Q 1/6841; B01L 7/52; B01L 2200/025; B01L 2300/0663; B01L 2300/12; B01L 2300/1894

(57) ABSTRACT

Provided herein are kits, systems and methods for analyzing analytes from a biological sample. The system includes an upper housing configured to receive a sample substrate and a thermal element configured to control a temperature of the sample substrate. The system includes a lower housing and an alignment mechanism coupling the upper housing to the lower housing. The alignment mechanism is configured to move the upper housing, the lower housing, or both into an arrangement having an open configuration, a closed configuration, or an intermediate configuration therebetween. The thermal element is configured to cool the sample substrate.

### CROSS-REFERENCE TO RELATED APPLICATIONS

This application claims the benefit of U.S. Provisional Application Ser. No. 63/714,559, filed Oct. 31, 2024. The contents of the related application are incorporated by reference in their entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provide substantial analyte data for dissociated tissue (i.e., single cells), but fail to provide information regarding the position of the single cell in a parent biological sample (e.g., tissue sample).

Spatial analysis of an analyte within a biological sample may require determining the sequence of the analyte sequence or a complement thereof and the sequence of the spatial barcode or a complement thereof to identify the location of the analyte. The biological sample may be placed on a solid support to improve specificity and efficiency when being analyzed for identification or characterization of an analyte, such as DNA, RNA or other genetic material, within the sample.

### SUMMARY

The present disclosure features systems, devices, and methods for sample handling. In particular implementations, the systems, devices, and methods described herein are configured to thermally condition (e.g., cool and/or heat) one or more sample substrates and/or sample support members that are configured to align sample substrates including biological samples to directly or indirectly contact array substrates including arrays of barcoded capture probes in spatial analysis.

An exemplary embodiment included herein is a system that can include one or more housings, e.g., upper and lower housings, configured to receive a sample substrate. The sample substrate can include a sample, such as a biological sample. The biological sample can be a tissue sample, such as tissue section, e.g., a fresh frozen tissue section. The system and/or device can include an alignment mechanism coupling the upper housing to the lower housing, or vice versa. The alignment mechanism can be configured to move the upper housing, the lower housing, or both into an arrangement having an open configuration, a closed configuration, or an intermediate configuration therebetween. The upper housing and/or the lower housing can include a thermal element configured to control a temperature of the sample substrate.

Such a system or device can include one or more of the following optional features. In some embodiments, the thermal element is mounted to the upper housing between the upper housing and the sample substrate. In some embodiments, the thermal element is mounted to the upper housing and surrounds the sample substrate. In some embodiments, the lower housing can include a lower housing thermal element that controls a temperature at a base support member of the lower housing. The thermal element can be configured to cool or heat the sample substrate. In some embodiments, the thermal element includes liquid nitrogen. In some embodiments, the thermal element can include a cooling liquid that circulates within the system to cool the sample substrate. The thermal element can optionally be transparent. Examples of a thermal element include, but are not limited to, a thermoelectric cooler (TEC) and a cooling plate. The thermal element can be configured to activate or deactivate based on the configuration of the system. For example, in the closed configuration, the thermal element can be configured to deactivate. The thermal element can be configured to, upon energizing, maintain a surface temperature of the sample substrate between −20° C. and 5° C. in the system. In some embodiments, the thermal element is configured to maintain a surface temperature of the sample substrate around 0° C. In some cases, the thermal element is configured to, upon energizing the thermal element, maintain a surface temperature of the sample substrate between 10° C. and 40° C. In some cases, the thermal element is configured to, upon energizing the thermal element in a cooling mode, maintain a surface temperature of the sample substrate between −80° C. and 5° C. (e.g., −20° C. and 5° C.). In some embodiments, the thermal element is configured to, upon energizing the thermal element in a heating mode, maintain a surface temperature of the sample substrate between 10° C. and 40° C. In some embodiments, the sample substrate can include a sample window. The upper housing can optionally include a light emitting diode (LED) assembly, wherein the LED assembly is configured to emit light through the sample window of the sample substrate. The LED assembly can be configured to emit light through the thermal element. The sample substrate can also include at least one base window. One or more of the base windows can configured to align with the sample window in the closed configuration.

An exemplary embodiment included herein is a method. The method can include connecting a substrate to an upper housing. The substrate can include a biological sample. The method can include cooling, by a thermal element of the upper housing, the substrate such that the biological sample is at or below a threshold temperature. In various embodiments, the thermal element can be configured to cool the substrate. The method can include aligning the upper housing with a lower housing by an alignment mechanism. The alignment mechanism can be configured to move the upper housing, the lower housing, or both into an arrangement having an open configuration, a closed configuration, or an intermediate configuration therebetween. In some embodiments, the methods described herein can include moving the upper housing towards the lower housing.

Such a method can include one or more of the following optional features. The method can include deactivating the thermal element at the upper housing; releasing one or more analytes from the biological sample; and capturing the one or more analytes from the biological sample onto a spatial array disposed on the lower housing. The method can including deactivating the thermal element responsive to moving the upper housing from the open configuration into the closed configuration. In some embodiments, the methods provided herein can align a base window at a base support member with a sample window at the sample support member. The methods herein can include maintaining a surface temperature of the sample substrate between −20° C. and 5° C. The threshold temperature can include a range of about −5° C. and 5° C., inclusive.

An exemplary embodiment included herein is a method for analyzing an analyte in a biological sample mounted on a first substrate. The method can include controlling a temperature of the first substrate in a cooling mode to maintain a fixed state of the biological sample on the first substrate. The method can include contacting the biological sample with at least one analyte capture agents, wherein an analyte capture agent comprises a capture agent barcode domain, wherein the capture agent barcode domain comprises an analyte binding moiety barcode and a capture handle sequence. The method can include aligning the first substrate with a second substrate comprising an array, such that at least a portion of the biological sample is aligned with at least a portion of the array, wherein the array comprises a plurality of capture probes. A capture probe of the plurality of capture probes can include: (i) a spatial barcode and (ii) a capture domain. The method can include, when the biological sample is aligned with at least a portion of the array, (i) releasing the capture agent barcode domain from the analyte by controlling the temperature of the first substrate in either a heating mode or an off mode; and (ii) passively or actively migrating the capture agent barcode domain from the biological sample to the array. The method can include coupling the capture handle sequence to the capture domain.

Any of the methods provided herein can include one or more of the following optional features. For example, the method can include determining (i) all or a part of the sequence of the capture agent barcode domain, or a complement thereof; and (ii) the sequence of the spatial barcode, or a complement thereof. The methods herein can optionally include using the determined sequence of (i) and (ii) to determine a location of the analyte in the biological sample. The determining step can include sequencing (i) all or a part of the capture agent barcode domain, or a complement thereof, and (ii) the sequence of the spatial barcode, or a complement thereof.

An exemplary embodiment included herein is a method for analyzing a nucleic acid analyte in a biological sample mounted on a first substrate. The method can include controlling a temperature of the first substrate in a cooling mode to maintain a fixed state of the biological sample on the first substrate. The method can include contacting the biological sample with a first probe and a second probe, wherein the first probe and second probe hybridize to the nucleic acid analyte, wherein the first probe and the second probe each comprise a sequence that is substantially complementary to a first and second sequence of the nucleic acid analyte, and wherein the second probe oligonucleotide comprises a capture probe binding domain. The method can include coupling the first probe and the second probe, thereby generating a connected probe. The method can include aligning the first substrate with a second substrate comprising an array, such that at least a portion of the biological sample is aligned with at least a portion of the array, wherein the array comprises a plurality of capture probes, wherein a capture probe of the plurality of capture probes comprises: (i) a spatial barcode and (ii) a capture domain. The method can include, when the biological sample is aligned with at least a portion of the array, (i) releasing the connected probe from the nucleic acid analyte by controlling the temperature of the first substrate in either a heating mode or an off mode; and (ii) passively or actively migrating the connected probe from the biological sample to the array. The method can include hybridizing the connected probe to the capture domain via the capture probe binding domain.

Such a method or any of the methods described herein can include one or more of the following optional features. For example, the first and second sequence of the nucleic acid analyte abut one another or are at least 1, 2, 3, 4, 5, 6, 7, 8, 9, 10, or more nucleotides away from one another. In some embodiments, the releasing step, the aligning step, and/or step (e) can include contacting the biological sample and the array with a reagent medium comprising a nuclease, a permeabilization agent, or a combination thereof. In some embodiments, the analyte comprises a protein or a fragment thereof, or a peptide. The releasing step can include simultaneously permeabilizing the biological sample and releasing the capture agent barcode domain from the analyte binding moiety, or simultaneously permeabilizing the biological sample and releasing the connected probe from the nucleic acid analyte. In some embodiments, the reagent medium includes a detergent. The detergent can be selected from sodium dodecyl sulfate (SDS), sarkosyl, saponin, polyethylene glycol tert-octylphenyl ether, or polysorbate 20. Further, the reagent medium can include less than 15 w/v % of a detergent selected from SDS and sarkosyl, or optionally the reagent medium includes as least 5% w/v % of a detergent selected from SDS and sarkosyl. In some embodiments, the reagent medium does not include a detergent and/or a permeabilization agent, preferably the reagent medium does not include sodium dodecyl sulfate (SDS) or sarkosyl. The reagent medium can further include one or more crowding agents, optionally PEG. In some embodiments, the coupling the first probe and the second probe comprises ligating the first probe and the second. In some embodiments, the ligating can include the use of a ligase, optionally wherein the ligase is selected from a T4 RNA ligase (Rnl2), a PBCV-1 DNA Ligase or Chlorella virus DNA Ligase, a single-stranded DNA ligase, and a T4 DNA ligase. The capture probe can include a poly(T) sequence. The capture probe can include a sequence complementary to the capture handle sequence or the capture probe binding domain. Further, the capture probe can include one or more functional domains, a unique molecular identifier (UMI), a cleavage domain, or a combination thereof. The analyte binding moiety can include an antibody. The analyte capture agent can include a linker, optionally wherein the linker is a cleavable linker. The cleavable linker can be a photo-cleavable linker, a UV-cleavable linker, or an enzyme cleavable linker. The nucleic acid analyte can be RNA or DNA, preferably the RNA is mRNA. Any of the methods provided herein can include an aligning step that includes: (i) mounting the first substrate on a first member of a support device, the first member configured to retain the first substrate; (ii) mounting the second substrate on a second member of the support device, (iii) applying the reagent medium to the first substrate and/or the second substrate, and (iv) operating an alignment mechanism of the support device to move the first member and/or the second member such that at least a portion of the biological sample is aligned with at least a portion of the array, and such that the portion of the biological sample and the portion of the array contact the reagent medium. In some embodiments, the alignment mechanism is coupled to the first member, the second member, or both the first member and the second member. In some embodiments, the alignment mechanism includes a linear actuator, optionally wherein: the linear actuator is configured to move the second member along an axis orthogonal to a plane or of the first member and/or the second member, and/or the linear actuator is configured to move the first member along an axis orthogonal to the plane of the first member and/or the second member, and/or the linear actuator is configured to move the first member, the second member, or both the first member and the second member at a velocity of at least 0.1 mm/sec, and/or the linear actuator is configured to move the first member, the second member, or both the first member and the second member with an amount of force of at least 0.1 lbs. Any of the methods provided herein can include, in the releasing step, a separation distance that is maintained between the first substrate and the second substrate, optionally wherein the separation distance is less than 50 microns, optionally wherein the separation distance is between 2-25 microns, optionally wherein the separation distance is measured in a direction orthogonal to a surface of the first substrate that supports the biological sample, and/or at least the portion of the biological sample is vertically aligned with at least a portion of the array. In some embodiments, at least one of the first substrate and the second substrate further includes a spacer, wherein after the first and second substrate being mounted on the support device, the spacer is disposed between the first substrate and second substrate and is configured to maintain the reagent medium within a chamber formed by the first substrate, the second substrate, and the spacer, and maintain a separation distance between the first substrate and the second substrate, the spacer positioned to at least partially surround an area on the first substrate on which the biological sample is disposed and/or the array disposed on the second substrate, wherein the area of the first substrate, the spacer, and the second substrate at least partially encloses a volume comprising the biological sample.

An exemplary embodiment included herein is a system or kit for analyzing an analyte in a biological sample. The system or kit can include: a support device configured to retain a first substrate and a second substrate, wherein a biological sample is placed on the first substrate, and wherein the second substrate comprises an array including a plurality of capture probes, wherein a capture probe of the plurality of capture probes comprises: (i) a spatial barcode and (ii) a capture domain. The system or kit can include at least one of (b1) a first probe and a second probe, wherein the first probe and the second probe each include a sequence that is substantially complementary to adjacent sequences of the analyte, wherein the second probe includes a capture probe binding domain, and wherein the first probe and the second probe are capable of being ligated together to form a connected probe; or (b2) a plurality of analyte capture agents, wherein an analyte capture agent of the plurality of analyte capture agents includes an analyte binding moiety and a capture agent barcode domain, wherein the analyte binding moiety specifically binds to the analyte in the biological sample, and wherein the capture agent barcode domain includes an analyte binding moiety barcode and an capture handle sequence. The system or kit can include a reagent medium that includes an agent for releasing the connected probe and optionally a permeabilization reagent. The system or kit can include instructions for performing any one of the methods described herein.

In any of the methods, systems, or kits provided herein one or more of the following optional features can be included. For example. the permeabilization agent can include a protease, optionally the protease is selected from trypsin, pepsin, elastase, or Proteinase K. The agent for releasing the connected probe can include an RNAse, optionally wherein the RNAse is selected from RNase A, RNase C, RNase H, or RNase I. The systems or kits disclosed herein can include an alignment mechanism on the support device to align the first substrate and the second substrate. The alignment mechanism can include a linear actuator, wherein the first substrate includes a first member and the second substrate comprises a second member. In some embodiments, the linear actuator is configured to move the second member along an axis orthogonal to a plane or of the first member and/or the second member, and/or the linear actuator is configured to move the first member along an axis orthogonal to the plane of the first member and/or the second member, and/or the linear actuator is configured to move the first member, the second member, or both the first member and the second member at a velocity of at least 0.1 mm/sec, and/or the linear actuator is configured to move the first member, the second member, or both the first member and the second member with an amount of force of at least 0.1 lbs.

Any one of the systems or kits provided herein can include one or more of the following optional features. an upper housing configured to receive a sample substrate and a thermal element; a lower housing; and an alignment mechanism coupling the upper housing to the lower housing. The alignment mechanism can be configured to move the upper housing, the lower housing, or both into an arrangement having an open configuration, a closed configuration, or an intermediate configuration therebetween. The thermal element can be configured to cool the sample substrate below room temperature. In some embodiments, the thermal element is configured to cool the sample substrate after insertion of the sample substrate into the upper housing. The thermal element can include a cooling plate in some embodiments. The sample substrate can include a sample window. The upper housing can include a light emitting diode (LED) assembly, wherein the LED assembly is configured to emit light through the sample window of the sample substrate. The LED assembly can be configured to emit light through the thermal element.

An exemplary embodiment included herein is a method. The method can include cooling, by a thermal element, a substrate comprising a biological sample such that the biological sample is at or below a threshold temperature. The method can include connecting a substrate to an upper housing. The method can include aligning the upper housing with a lower housing by an alignment mechanism, the alignment mechanism being configured to move the upper housing, the lower housing, or both into an arrangement having an open configuration, a closed configuration, or an intermediate configuration therebetween. The method can include moving the upper housing towards the lower housing; wherein the thermal element is configured to cool the substrate.

Such a method can include one or more of the following optional features. The method can include connecting the thermal element to the upper housing. The thermal element can include a cooling plate.

An exemplary embodiment included herein is a system. The system can include a thermal element, an upper housing configured to receive a sample substrate, a lower housing, and an alignment mechanism coupling the upper housing to the lower housing. The alignment mechanism can be configured to move the upper housing, the lower housing, or both into an arrangement having an open configuration, a closed configuration, or an intermediate configuration therebetween. The thermal element can be configured to cool the sample substrate before insertion of the sample substrate into the upper housing.

Particular implementations can, in certain instances, realize one or more of the following advantages. First, the systems, devices, and methods described herein facilitate the capture of targets (e.g., RNA, DNA, protein, etc.) in tissue samples while retaining spatial information for spatial assays. For example, target fixation and release can be facilitated by temperature control of a substrate that holds the sample (e.g., a sample substrate). Second, in some embodiments, targets can be fixed, captured, and released without chemical fixation. For example, the systems, devices, and methods described herein facilitate target fixation by controlling the temperature of at least one of the sample substrate, the sample, or both the sample substrate and the sample throughout the capturing process (e.g., before, during, and after capturing). Third, the systems, devices, and methods described herein facilitate a high assay sensitivity result that includes predictable and controllable target releases. Fourth, systems, devices, and methods described herein facilitate additional target and sample processing (e.g., staining, manipulation, etc.) after capturing the assay. For example, additional target and sample processing can be performed after capturing the assay because the sample has not yet undergone digestion and remains intact during the temperature control process.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows and exemplary workflow for performing templated capture and producing a ligation product, and FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIG. 12 is a perspective view of an exemplary embodiment of a sample handling system as described herein.

FIG. 13 is a perspective view of a lower housing of the sample handling system of FIG. 12.

FIG. 14 is a perspective view of the lower housing of the sample handling system of FIG. 12 with a housing cover removed.

FIG. 15 illustrates angled closure of a sample support member and a base member of the sample handling system described herein.

FIG. 16A is a top view of the lower housing of the sample handling system of FIG. 14 with a shroud and the housing cover removed.

FIG. 16B is a perspective view of the lower housing of the sample handling system of FIG. 14 with a shroud and the housing cover removed.

FIG. 17 is a perspective view of an upper housing of the sample handing system of FIG. 12.

FIG. 18 is a top view of an assembly of components of the upper housing of FIG. 17.

FIG. 19 is a perspective view of the portion of components of FIG. 17 with the linear motion member removed.

FIG. 20 is side view of the assembly of components of FIG. 17.

FIG. 21A shows an exemplary sandwiching process where a first substrate (e.g., a slide), including a biological sample, and a second substrate (e.g., array slide) are brought into proximity with one another.

FIG. 21B shows a fully formed sandwich configuration creating a chamber formed from the one or more spacers, the first substrate, and the second substrate.

FIG. 22 is a schematic diagram showing two substrates supporting a sample and a feature array, respectively.

FIG. 23 shows an example analytical workflow using dried permeabilization reagents.

FIG. 24 shows an example analytical workflow using temperature-controlled first and second members of an example sample holder.

FIG. 25 shows an exemplary embodiment of a sample handling system as described herein.

Like reference symbols in the various drawings indicate like elements.

### DETAILED DESCRIPTION

### A. Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; and the Visium Spatial Gene Expression Reagent Kits User Guide (e.g., Rev F, dated January 2022) and/or the Visium Spatial Gene Expression Reagent Kits—Tissue Optimization User Guide (e.g., Rev E, dated February 2022), both of which are available at the 10× Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I)(b) of PCT Publication No. WO2020/176788 and/or U.S. Patent Application Publication No. 2020/0277663, which is herein incorporated by reference. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays may be paraffin blocks produced by extracting cylindrical tissue cores from different paraffin donor blocks and re-embedding these tissue cores into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archaeon; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example, methanol. In some embodiments, instead of methanol, acetone or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample, e.g., using acetone and/or alcohol (e.g., methanol, acetone-methanol), is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing, e.g., by formalin or PFA, the biological sample is dehydrated via sucrose gradient. In some instances, the fixed biological sample is treated with a sucrose gradient and then embedded in a matrix, e.g., OCT compound. In some instances, the fixed biological sample is not treated with a sucrose gradient, but rather is embedded in a matrix, e.g., OCT compound after fixation. In some embodiments when a fixed frozen tissue sample is treated with a sucrose gradient, the sample can be rehydrated using an ethanol gradient. In some embodiments, the PFA or formalin fixed biological sample, which can be optionally dehydrated via sucrose gradient and/or embedded in OCT compound, is then frozen, e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated using an ethanol gradient.

In some instances, the biological sample (e.g., a fixed frozen tissue sample) is treated with a citrate buffer. Citrate buffer can be used to decrosslink antigens and fixation medium for antigen retrieval in the biological sample. Thus, any suitable decrosslinking agent can be used in addition, or alternatively, to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, the sample is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, an acid, and a soluble organic compound that preserves morphology and biomolecules. PAXgene provides a two-reagent fixative system in which tissue is firstly fixed in a solution containing methanol and acetic acid, then stabilized in a solution containing ethanol. See, Ergin B. et al., J Proteome Res. 2010 Oct. 1; 9(10):5188-96; Kap M. et al., PLoS One.; 6(11):e27704 (2011); and Mathieson W. et al., Am J Clin Pathol.; 146(1):25-40 (2016), each of which is hereby incorporated by reference in its entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample, is fixed, for example in methanol, acetone, acetone-methanol, PFA, PAXgene, or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than of a fresh sample, thereby capturing RNA directly from fixed samples, e.g., by capture of a common sequence such as a poly(A) tail of an mRNA molecule, can be more difficult. By utilizing RTL probes that hybridize to RNA target sequences in the transcriptome, RNA analytes can be captured without requiring that both a poly(A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I)(d) of PCT Publication No. WO2020/176788 and/or U.S. Patent Application Publication No. 2020/0277663, which is herein incorporated by reference.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes, as disclosed herein, to the biological sample.

In some embodiments, the methods include staining the biological sample. In some embodiments, the staining includes the use of hematoxylin and/or eosin. Non-limiting examples of stains include histological stains (e.g., hematoxylin and/or eosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI (4′,6-diamidino-2-phenylindole), eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663, which is herein incorporated by reference. Briefly, any of the methods described herein includes permeabilizing the biological sample. For example, the biological sample can be permeabilized to facilitate transfer of extension products to the capture probes on the array. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, polysorbate 20, N-lauroylsarcosine sodium salt solution, saponin, Triton X-100™, Tween-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, which is herein incorporated by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663, which is herein incorporated by reference. Generation of capture probes can be achieved by any appropriate method, including those described in Section (II)(d)(ii) of PCT Publication No. WO2020/176788 and/or U.S. Patent Application Publication No. 2020/0277663, which is herein incorporated by reference.

In some instances, a capture probe and a nucleic acid analyte interaction (or any other nucleic acid to nucleic acid interaction) occurs because the sequences of the two nucleic acids are substantially complementary to one another. By “substantial,” “substantially,” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues of the other nucleic acid sequence. Sequences are said to be “substantially complementary” when at least 60% (e.g., at least 70%, at least 80%, or at least 90%) of the residues of one nucleic acid sequence are complementary to residues of the other nucleic acid sequence. In some embodiments, the biological sample is mounted on a first substrate and the substrate comprising the array of capture probes is a second substrate. In this configuration, one or more analytes or analyte derivatives (e.g., intermediate agents; e.g., ligation products) are then released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described, e.g., in U.S. Patent Application Pub. No. 2021/0189475 and PCT Pub. Nos. WO 2021/252747 A1, WO 2022/061152 A2, and WO 2022/140028 A1, each of which is herein incorporated by reference.

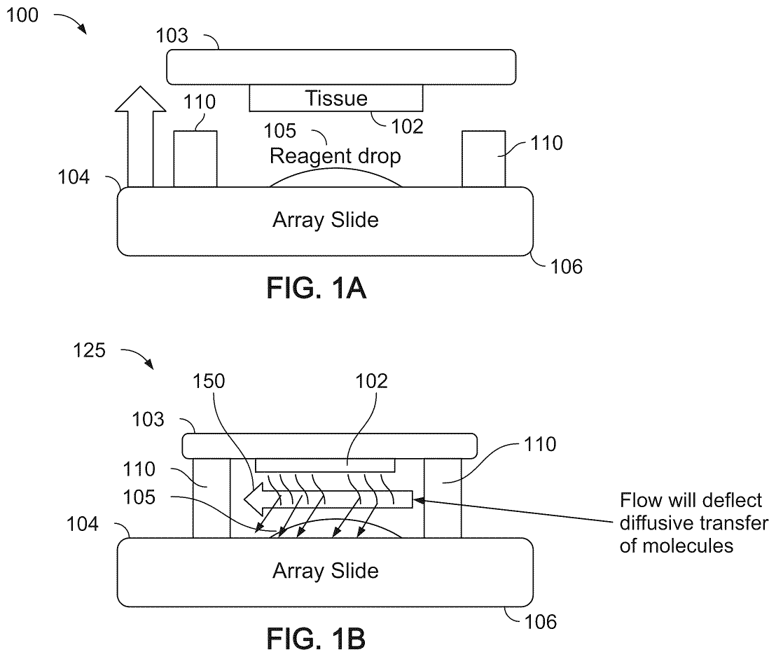

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A, a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788 and U.S. Patent Application Pub. No. 2021/0189475, each of which is hereby incorporated by reference in its entirety.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns (μm) and about 1 mm (e.g., between about 2 μm and about 800 μm, between about 2 μm and about 700 μm, between about 2 μm and about 600 μm, between about 2 μm and about 500 μm, between about 2 μm and about 400 μm, between about 2 μm and about 300 μm, between about 2 μm and about 200 μm, between about 2 μm and about 100 μm, between about 2 μm and about 25 μm, or between about 2 μm and about 10 μm), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 μm. In some embodiments, the separation distance is less than 50 μm. In some embodiments, the separation distance is less than 25 μm. In some embodiments, the separation distance is less than 20 μm. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate (e.g., slide 103), and the second substrate (e.g., slide 104) may reduce or prevent undesirable movement (e.g., convective movement) of transcripts and/or molecules during the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., U.S. Patent Application Pub. No. 2021/0189475 and PCT Publ. No. WO 2022/061152 A2, each of which is incorporated by reference in its entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

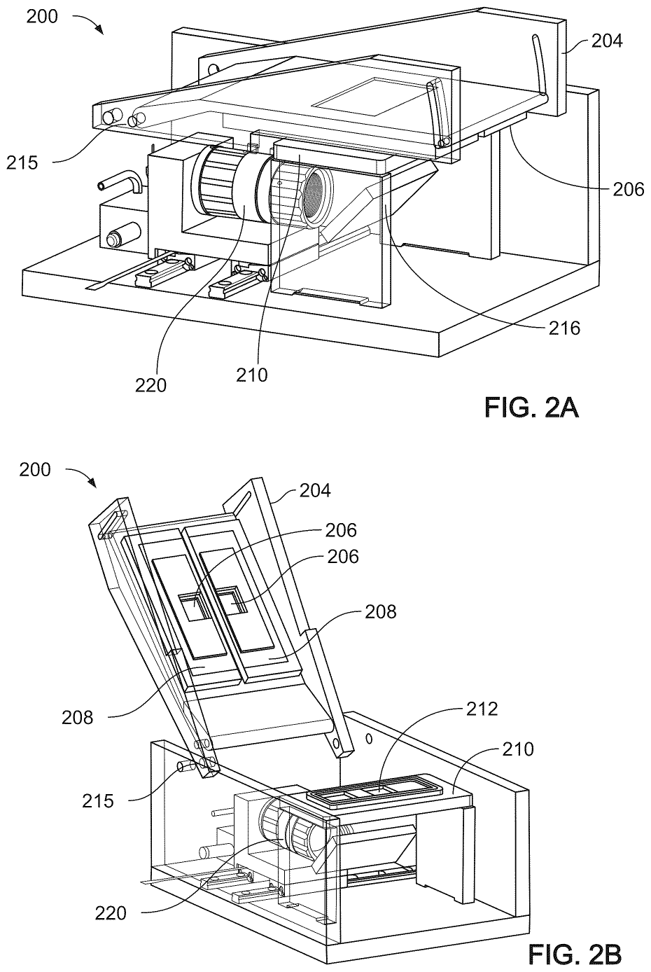

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

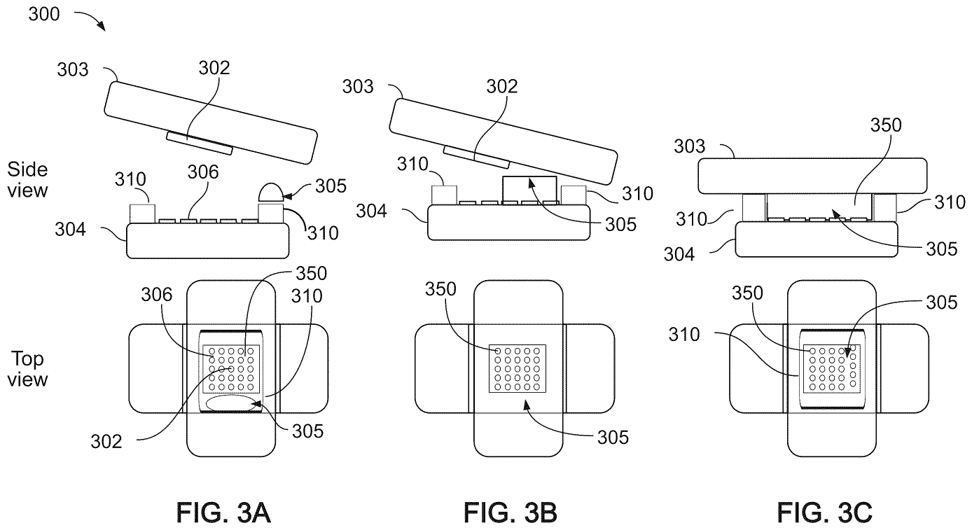

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right-hand side of side view, it should be understood that such depiction is not meant to be limiting as to the location of the reagent medium on the spacer.

FIG. 3B shows that as the first substrate lowers and/or as the second substrate rises, the dropped side of the first substrate (e.g., a side of the slide 303 angled toward the slide 304) may contact the reagent medium 305. The dropped side of the slide 303 may urge the reagent medium 305 toward the opposite direction (e.g., towards an opposite side of the spacer 310, towards an opposite side of the slide 303 relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

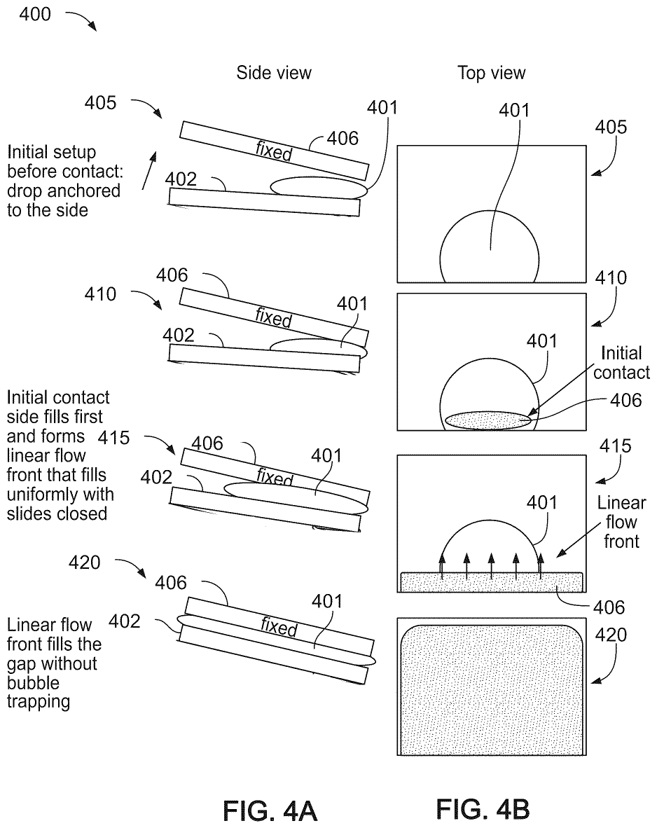

FIG. 4A is a side view of the angled closure workflow 400 in accordance with some exemplary implementations. FIG. 4B is a top view of the angled closure workflow 400 in accordance with some exemplary implementations. As shown at step 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills the gap between the two substrates 406 and 402 uniformly with the slides closed.

At step 415, the substrate 406 is further lowered toward the substrate 402 (or the substrate 402 is raised up toward the substrate 406) and the dropped side of the substrate 406 may contact and urge the reagent medium toward the side opposite the dropped side, thereby creating a linear or low curvature flow front that may prevent or reduce bubble trapping between the substrates.

At step 420, the reagent medium 401 fills the gap between the substrate 406 and the substrate 402. The linear flow front of the liquid reagent may be formed by squeezing the reagent medium 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™, SDS), and enzymes (e.g., trypsin or other proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and SDS. More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of SDS or a sodium salt thereof, proteinase K, pepsin, N-lauroylsarcosine, and RNase.

In some embodiments, the reagent medium comprises polyethylene glycol (PEG). In some embodiments, the PEG molecular weight is from about 2K to about 16K. In some embodiments, the PEG is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes, which is herein incorporated by reference). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to a terminus (e.g., a 3′ or 5′ end) of the capture probe, thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Publication No. WO2020/176788 and/or U.S. Patent Application Publication No. 2020/0277663, which is herein incorporated by reference. Analysis of captured analytes (and/or intermediate agents or portions thereof), for example, including sample removal, extension of capture probes using the captured analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663, which is herein incorporated by reference. Some quality control measures are described in Section (II)(h) of PCT Publication No. WO2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference in its entirety.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of PCT Publication No. WO2020/176788 and/or U.S. Patent Application Publication No. 2020/0277663, which is herein incorporated by reference. Exemplary features and geometric attributes of an array can be found in Sections (II)(d)(i), (II)(d)(iii), and (II)(d)(iv) of PCT Publication No. WO2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads or wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of PCT Publication No. WO2020/176788 and/or U.S. Patent Application Publication No. 2020/0277663, which is herein incorporated by reference.

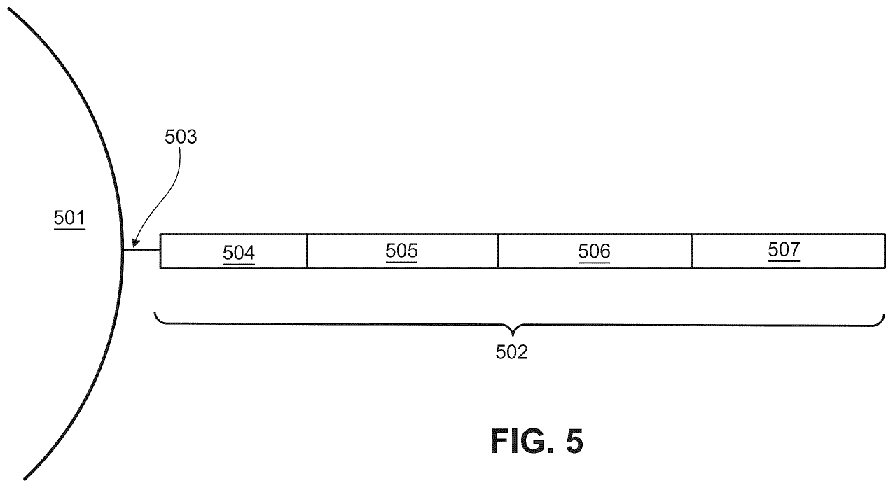

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

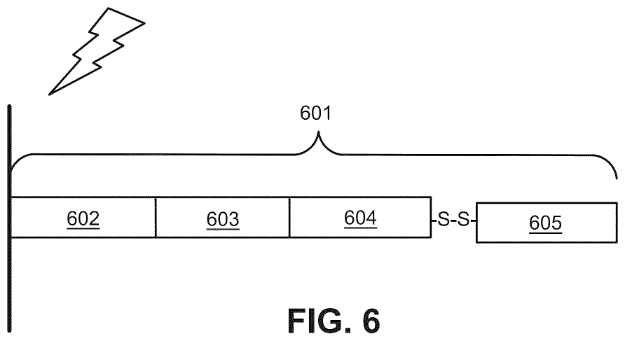

FIG. 6 is a schematic illustrating a cleavable capture probe, wherein the cleaved capture probe can enter into a non-permeabilized cell and bind to analytes within the cell. The capture probe 601 can contain a cleavage domain 602, a cell penetrating peptide 603, a reporter molecule 604, and a disulfide bond (—S—S—). 605 represents all other parts of a capture probe, for example, a spatial barcode and a capture domain.

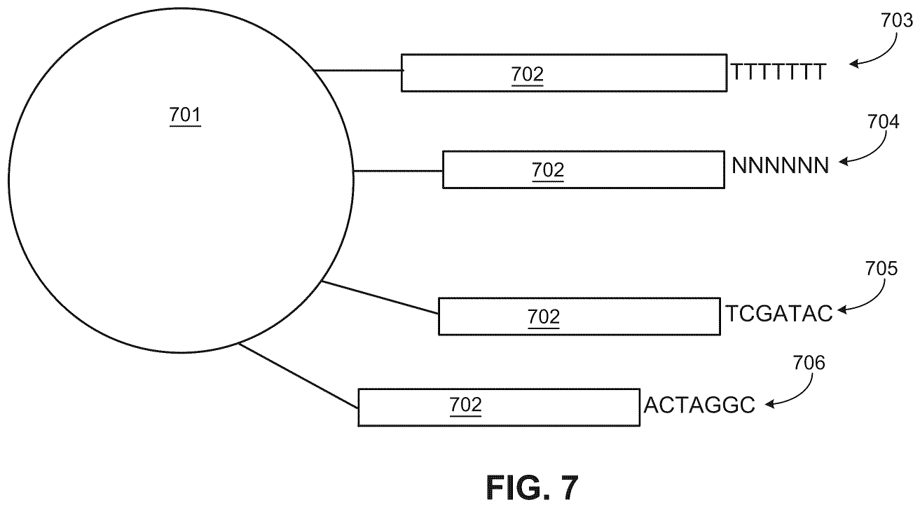

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature can include the spatial barcode 702 in combination with a poly(T) capture domain 703, designed to capture mRNA target analytes. A second type of capture probe associated with the feature can include the spatial barcode 702 in combination with a random N-mer capture domain 704 for gDNA analysis. A third type of capture probe associated with the feature can include the spatial barcode 702 in combination with a capture domain complementary to the analyte capture agent of interest 705. A fourth type of capture probe associated with the feature can include the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and/or metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature) change, or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequence 504 are common to all of the probes attached to a given feature. In some embodiments, the UMI sequence 506 of a capture probe attached to a given feature is different from the UMI sequence of a different capture probe attached to the given feature.

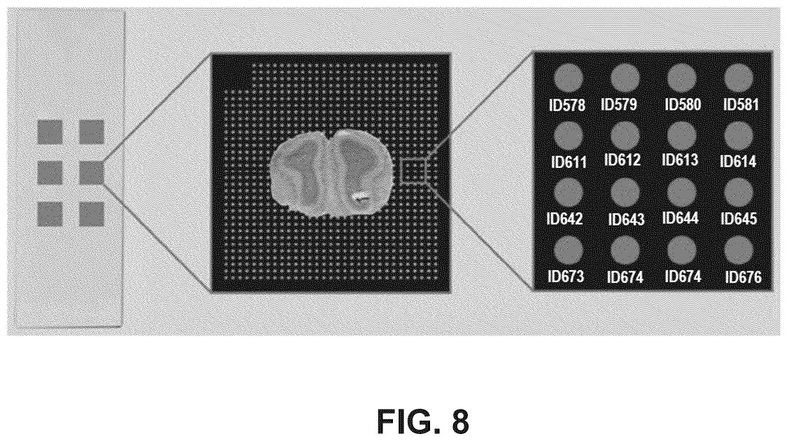

FIG. 8 depicts an exemplary arrangement of barcoded features within an array. From left to right, FIG. 8 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (e.g., labelled as ID578, ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128, which is herein incorporated by reference in its entirety. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture probe binding domain (e.g., a poly(A) sequence or a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location, and optionally, the abundance of the analyte in the biological sample.

In some instances, one or both of the oligonucleotides may hybridize to genomic DNA (gDNA), which can lead to false positive sequencing data from ligation events on gDNA (off target) in addition to the desired (on target) ligation events on target nucleic acids (e.g., mRNA). Thus, in some embodiments, the disclosed methods can include contacting the biological sample with a deoxyribonuclease (DNase). The DNase can be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

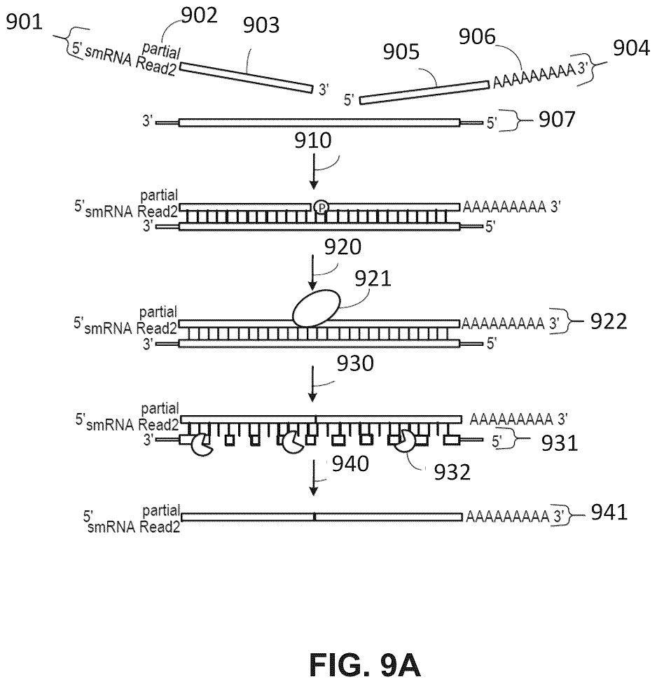

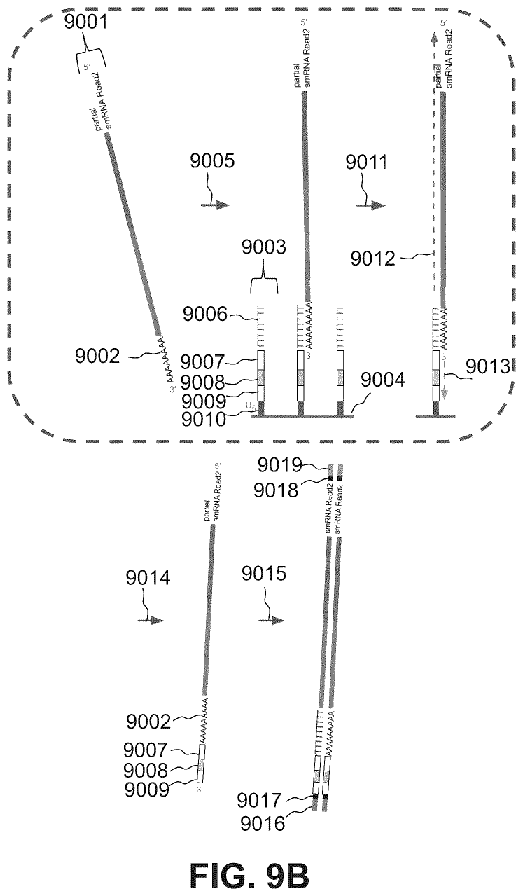

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly(A) sequence) 906, the first probe 901 and the second probe 904 hybridize 910 to an analyte 907. A ligase 921 ligates 920 the first probe 901 to the second probe 904, thereby generating a ligation product 922. The ligation product 922 is then released 930 from the analyte 931 by digesting the analyte 907 using an endoribonuclease 932. The sample is permeabilized 940 and the ligation product 941 is able to hybridize to a capture probe on the substrate. Methods and compositions for spatial detection using templated ligation have been described in PCT Publication. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product 9001 specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the ligation products (i.e., compared to no permeabilization). In some embodiments, polymerization (e.g., reverse transcription (RT)) reagents can be added to permeabilized biological samples. Incubation with the polymerization reagents can be used to extend the capture probes 9011 to produce spatially-barcoded full-length cDNA 9012 and 9013 from the captured ligation products (e.g., ligation products). The ligation products can be extended using the capture probe as a template to include a complement of the capture probe, thereby generating extended ligation products.

In some embodiments, the extended ligation products can be denatured 9014, released from the capture probe, and transferred (e.g., to a clean tube) for amplification and/or library construction. The spatially-barcoded ligation products can be amplified 9015 via PCR prior to library construction. P5 9016, i5 9017, i7 9018, and P7 9019 sequences can be used as sample indexes. The amplicons can then be sequenced using paired-end sequencing using TruSeq Read 1 and TruSeq Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663, which is herein incorporated by reference.

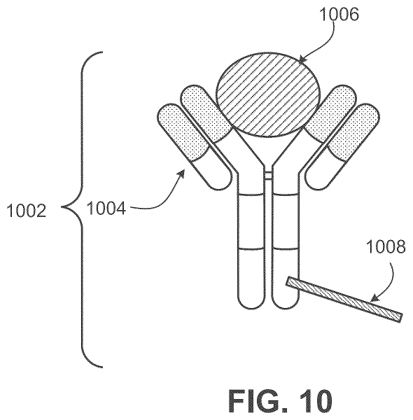

FIG. 10 is a schematic diagram of an exemplary analyte capture agent 1002 comprised of an analyte binding moiety 1004 and an analyte-binding moiety barcode domain 1008. The exemplary analyte binding moiety 1004 is capable of binding to an analyte 1006 and the analyte capture agent 1002 is capable of interacting with a spatially-barcoded capture probe. The analyte binding moiety 1004 can bind to the analyte 1006 with high affinity and/or with high specificity. The analyte capture agent 1002 can include: (i) an analyte binding moiety barcode domain 1008, which serves to identify the analyte binding moiety, and (ii) an analyte capture sequence, which can hybridize to at least a portion or an entirety of a capture domain of a capture probe. The analyte binding moiety 1004 can include a polypeptide and/or an aptamer. The analyte binding moiety 1004 can include an antibody or antibody fragment (e.g., an antigen-binding fragment).

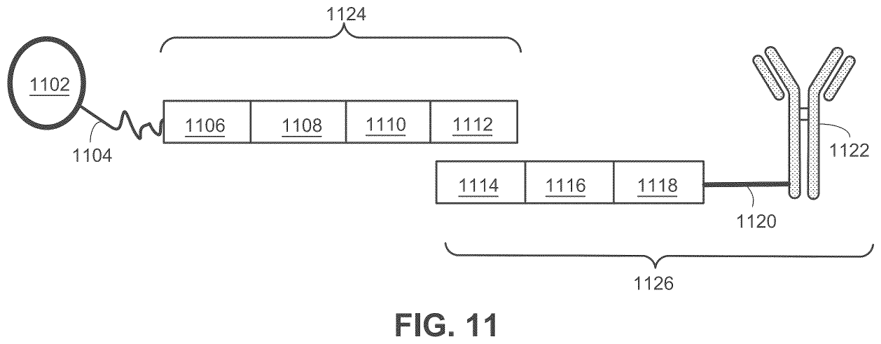

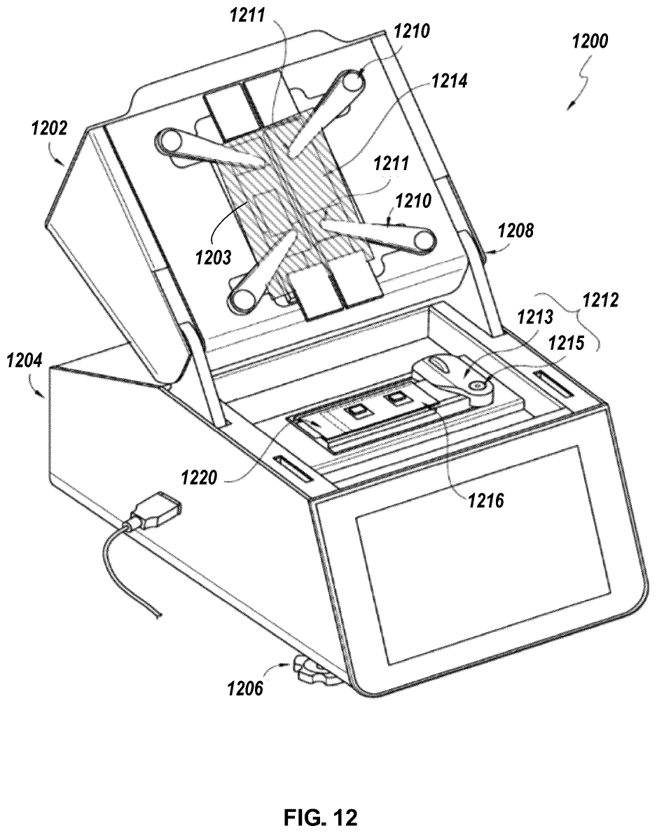

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequence 1106 and a UMI 1110, as described elsewhere herein. The capture probe can be affixed 1104 to a feature such as a bead 1102. The capture probe 1124 can also include a capture domain 1112 that is capable of binding to an analyte capture agent 1126. The analyte binding moiety barcode domain of the analyte capture agent 1126 can include functional sequence 1118, analyte binding moiety barcode 1116, and an analyte capture sequence 1114 that is capable of binding (e.g., hybridizing) to the capture domain 1112 of the capture probe 1124. The analyte capture agent 1126 can also include a linker 1120 that allows the analyte binding moiety barcode domain (e.g., including the functional sequence 1118, analyte binding moiety barcode 1116, and analyte capture sequence 1114) to couple to the analyte binding moiety 1122. In some embodiments, the linker 1120 is a cleavable linker. In some embodiments, the cleavable linker is a photo-cleavable linker, a UV-cleavable linker, chemical-cleavable, thermal-cleavable, or an enzyme cleavable linker. In some instances, the cleavable linker is a disulfide linker. A disulfide linker can be cleaved by use of a reducing agent, such as dithiothreitol (DTT), beta-mercaptoethanol (BME), or Tris (2-carboxyethyl) phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the captured analytes are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that each spatial barcode is uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location or a fiducial marker) of the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663, which is herein incorporated by reference. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022) and/or the Visium Spatial Gene Expression Reagent Kits—Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II)(e)(ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320, which is herein incorporated by reference.

Suitable systems for performing spatial analysis can include components such as a chamber (e.g., a flow cell or a sealable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in its entirety.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in its entirety. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### B. Methods, Compositions, Devices, and Systems for Analyzing Analytes and Derivatives Thereof in Biological Samples

### i. (a) Introduction

Provided herein are methods, devices, compositions, and systems for analyzing the location of an analyte (e.g., a nucleic acid and/or protein analyte) in a biological sample. In some instances, the methods include aligning (e.g.,, sandwiching) a first substrate having the biological sample with a second substrate that includes a plurality of capture probes, thereby “sandwiching” the biological sample between the two substrates. Upon aligning the biological sample with the substrate having a plurality of capture probes, the location of an analyte (e.g., a nucleic acid and/or protein analyte) in a biological sample can be determined, as provided herein. The methods include an advantage in that steps provided herein prior to analyte or analyte-derived molecule by the capture probe, most—if not all—steps can be performed on a substrate that does not have capture probes, thereby providing a method that is cost effective.

The methods and systems provided herein can be applied to an analyte or an analyte-derived molecule(s). As used herein, an analyte derived molecule includes, without limitation, a connected probe (e.g., a ligation product) from an RNA-templated ligation (RTL) assay, a product of reverse transcription (e.g., an extended capture probe or a complement thereof, a first or second strand cDNA), and an analyte binding moiety barcode (e.g., a binding moiety barcode that identifies that analyte binding moiety (e.g., an antibody)). In some embodiments, the analyte or analyte derived molecules comprise RNA and/or DNA. In some embodiments, the analyte or analyte derived molecules comprise one or more proteins.

In some instances, the methods, devices, compositions, and systems disclosed herein provide efficient release of an analyte or analyte derived molecule from a biological sample so that it can be easily captured or detected using methods disclosed herein.

Embodiments of the methods, devices, compositions, and systems disclosed herein are provided below.

### (A) Exemplary Biological Samples

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section. In some embodiments, the tissue is flash-frozen and sectioned. Any suitable methods described herein or known in the art can be used to flash-freeze and section the tissue sample. In some embodiments, the biological sample, e.g., the tissue, is flash-frozen using liquid nitrogen before sectioning. In some embodiments, the sectioning is performed using cryosectioning. In some embodiments, the methods further comprises a thawing step, after the cryosectioning. In some embodiments, the biological sample, e.g., the tissue sample is fixed, for example in methanol, acetone, PFA or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RTL methods disclosed herein. A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probe oligonucleotides that hybridize to RNA target sequences in the transcriptome, one can avoid a requirement for RNA analytes to have both a poly(A) tail and target sequences intact. Accordingly, RTL probes can be utilized to beneficially improve capture and spatial analysis of fixed samples.

The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to deaminating the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

As discussed previously, a tissue sample can be obtained from any suitable location in a tissue or organ of a subject, e.g., a human subject. In some instances, the sample is a human sample. In some instances, the sample is a human breast tissue sample. In some instances, the sample is a human brain tissue sample. In some embodiments, the sample is an embryo sample. The embryo sample can be a non-human embryo sample. In some instances, the sample is a mouse embryo sample.

### (B) Exemplary First and Second Substrates

In some instances, the biological sample is placed (e.g., mounted or otherwise immobilized) on a first substrate. The first substrate can be any solid or semi-solid support upon which a biological sample can be mounted. In some instances, the first substrate is a slide. In some instances, the slide is a glass slide. In some embodiments, the first substrate is made of glass, silicon, paper, hydrogel, polymer monoliths, or other material known in the art. In some embodiments, the first substrate is comprised of an inert material or matrix (e.g., glass slides) that has been functionalized by, for example, treating the substrate with a material comprising reactive groups which facilitate mounting of the biological sample.

In some embodiments, the first substrate does not comprise a plurality (e.g., array) of capture probes, each comprising a spatial barcode.

A substrate, e.g., a first substrate and/or a second substrate, can generally have any suitable form or format. For example, a substrate can be flat, curved, e.g., convexly or concavely curved. For example, a first substrate can be curved towards the area where the interaction between a biological sample, e.g., tissue sample, and a first substrate takes place. In some embodiments, a substrate is flat, e.g., planar, chip, or slide. A substrate can contain one or more patterned surfaces within the first substrate (e.g., channels, wells, projections, ridges, divots, etc.).

A substrate, e.g., a first substrate and/or second substrate, can be of any desired shape. For example, a substrate can be typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments wherein a substrate structure is flat, the substrate structure can be any appropriate type of support having a flat surface (e.g., a chip or a slide such as a microscope slide).

First and/or second substrates can optionally include various structures such as, but not limited to, projections, ridges, and channels. A substrate can be micropatterned to limit lateral diffusion of analytes (e.g., to improve resolution of the spatial analysis). A substrate modified with such structures can be modified to allow association of analytes, features (e.g., beads), or probes at individual sites. For example, the sites where a substrate is modified with various structures can be contiguous or non-contiguous with other sites.

In some embodiments, the surface of a first and/or second substrate is modified to contain one or more wells, using techniques such as (but not limited to) stamping, microetching, or molding techniques. In some embodiments in which a first and/or second substrate includes one or more wells, the first substrate can be a concavity slide or cavity slide. For example, wells can be formed by one or more shallow depressions on the surface of the first and/or second substrate. In some embodiments, where a first and/or second substrate includes one or more wells, the wells can be formed by attaching a cassette (e.g., a cassette containing one or more chambers) to a surface of the first substrate structure.

In some embodiments where the first and/or second substrate is modified to contain one or more structures, including but not limited to, wells, projections, ridges, features, or markings, the structures can include physically altered sites. For example, a first and/or second substrate modified with various structures can include physical properties, including, but not limited to, physical configurations, magnetic or compressive forces, chemically functionalized sites, chemically altered sites, and/or electrostatically altered sites. In some embodiments where the first substrate is modified to contain various structures, including but not limited to wells, projections, ridges, features, or markings, the structures are applied in a pattern. Alternatively, the structures can be randomly distributed.

In some embodiments, a first substrate includes one or more markings on its surface, e.g., to provide guidance for aligning at least a portion of the biological sample with a plurality of capture probes on the second substrate during a sandwich process disclosed herein. For example, the first substrate can include a sample area indicator identifying the sample area. In some embodiments, during a sandwiching process described herein the sample area indicator on the first substrate is aligned with an area of the second substrate comprising a plurality of capture probes. In some embodiments, the first and/or second substrate can include a fiducial mark. In some embodiments, the first and/or second substrate does not comprise a fiducial mark. In some embodiments, the first substrate does not comprise a fiducial mark and the second substrate comprises a fiducial mark. Such markings can be made using techniques including, but not limited to, printing, sand-blasting, and depositing on the surface.

In some embodiments, imaging can be performed using one or more fiducial markers, i.e., objects placed in the field of view of an imaging system which appear in the image produced. Fiducial markers are typically used as a point of reference or measurement scale. Fiducial markers can include, but are not limited to, detectable labels such as fluorescent, radioactive, chemiluminescent, and colorimetric labels. The use of fiducial markers to stabilize and orient biological samples is described, for example, in Carter et al., Applied Optics 46:421-427, 2007), the entire contents of which are incorporated herein by reference. In some embodiments, a fiducial marker can be a physical particle (e.g., a nanoparticle, a microsphere, a nanosphere, a bead, a post, or any of the other exemplary physical particles described herein or known in the art).

In some embodiments, a fiducial marker can be present on a first substrate to provide orientation of the biological sample. In some embodiments, a microsphere can be coupled to a first substrate to aid in orientation of the biological sample. In some examples, a microsphere coupled to a first substrate can produce an optical signal (e.g., fluorescence). In some embodiments, a quantum dot can be coupled to the first substrate to aid in the orientation of the biological sample. In some examples, a quantum dot coupled to a first substrate can produce an optical signal.

In some embodiments, a fiducial marker can be an immobilized molecule with which a detectable signal molecule can interact to generate a signal. For example, a marker nucleic acid can be linked or coupled to a chemical moiety capable of fluorescing when subjected to light of a specific wavelength (or range of wavelengths). Although not required, it can be advantageous to use a marker that can be detected using the same conditions (e.g., imaging conditions) used to detect a labelled cDNA.

In some embodiments, a fiducial marker can be randomly placed in the field of view. For example, an oligonucleotide containing a fluorophore can be randomly printed, stamped, synthesized, or attached to a first substrate (e.g., a glass slide) at a random position on the first substrate. A tissue section can be contacted with the first substrate such that the oligonucleotide containing the fluorophore contacts, or is in proximity to, a cell from the tissue section or a component of the cell (e.g., an mRNA or DNA molecule). An image of the first substrate and the tissue section can be obtained, and the position of the fluorophore within the tissue section image can be determined (e.g., by reviewing an optical image of the tissue section overlaid with the fluorophore detection). In some embodiments, fiducial markers can be precisely placed in the field of view (e.g., at known locations on a first substrate). In this instance, a fiducial marker can be stamped, attached, or synthesized on the first substrate and contacted with a biological sample. Typically, an image of the sample and the fiducial marker is taken, and the position of the fiducial marker on the first substrate can be confirmed by viewing the image.

In some embodiments, a fiducial marker can be an immobilized molecule (e.g., a physical particle) attached to the first substrate. For example, a fiducial marker can be a nanoparticle, e.g., a nanorod, a nanowire, a nanocube, a nanopyramid, or a spherical nanoparticle. In some examples, the nanoparticle can be made of a heavy metal (e.g., gold). In some embodiments, the nanoparticle can be made from diamond. In some embodiments, the fiducial marker can be visible by eye.

A wide variety of different first substrates can be used for the foregoing purposes. In general, a first substrate can be any suitable support material. Exemplary first substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene polycarbonate, or combinations thereof.

Among the examples of first substrate materials discussed above, polystyrene is a hydrophobic material suitable for binding negatively charged macromolecules because it normally contains few hydrophilic groups. For nucleic acids immobilized on glass slides, by increasing the hydrophobicity of the glass surface the nucleic acid immobilization can be increased. Such an enhancement can permit a relatively more densely packed formation (e.g., provide improved specificity and resolution).

In another example, a first substrate can be a flow cell. Flow cells can be formed of any of the foregoing materials, and can include channels that permit reagents, solvents, features, and analytes to pass through the flow cell. In some embodiments, a hydrogel embedded biological sample is assembled in a flow cell (e.g., the flow cell is utilized to introduce the hydrogel to the biological sample). In some embodiments, a hydrogel embedded biological sample is not assembled in a flow cell. In some embodiments, the hydrogel embedded biological sample can then be prepared and/or isometrically expanded as described herein.

Exemplary substrates similar to the first substrate (e.g., a substrate having no capture probes) and/or the second substrate are described in Section (I) above and in WO 2020/123320, which is hereby incorporated by reference in its entirety.

### (b) Capturing Nucleic Acid Analytes Using RNA-Templated Ligation

In some embodiments, the methods, devices, compositions, and systems described herein utilize RNA-templated ligation to detect the analyte. As used herein, spatial “RNA-templated ligation,” or “RTL” or simply “templated ligation” is a process wherein individual probe oligonucleotides (e.g., a first probe oligonucleotide, a second probe oligonucleotide) in a probe pair hybridize to adjacent sequences of an analyte (e.g., an RNA molecule) in a biological sample (e.g., a tissue sample). The RTL probe oligonucleotides are then coupled (e.g., ligated) together, thereby creating a connected probe (e.g., a ligation product). RNA-templated ligation is disclosed in PCT Publ. No. WO 2021/133849 A1 and US Publ. No. US 2021/0285046 A1, each of which is incorporated by reference in its entirety.

An advantage to using RTL is that it allows for enhanced detection of analytes (e.g., low expressing analytes) because both probe oligonucleotides must hybridize to the analyte in order for the coupling (e.g., ligating) reaction to occur. As used herein, “coupling” refers to an interaction between two probe oligonucleotides that results in a single connected probe that comprises the two probe oligonucleotides. In some instances, coupling is achieved through ligation. In some instances, coupling is achieved through extension of one probe oligonucleotide to the second probe oligonucleotide followed by ligation. In some instances, coupling is achieved through hybridization (e.g., using a third probe oligonucleotide that hybridized to each of the two probe oligonucleotides) followed by extension of one probe oligonucleotide or gap filling of the sequence between the two probe oligonucleotides using the third probe oligonucleotide as a template.

The connected probe (e.g., ligation product) that results from the coupling (e.g., ligation) of the two probe oligonucleotides can serve as a proxy for the target analyte, as such an analyte derived molecule. Further, it is appreciated that probe oligonucleotide pairs can be designed to cover any gene of interest. For example, a pair of probe oligonucleotides can be designed so that each analyte, e.g., a whole exome, a transcriptome, a genome, can conceivably be detected using a probe oligonucleotide pair.

In some instances, disclosed herein are methods for analyzing an analyte in a biological sample comprising (a) hybridizing a first probe oligonucleotide and a second probe oligonucleotide to the analyte, wherein the first probe oligonucleotide and the second probe oligonucleotide each comprise a sequence that is substantially complementary to adjacent sequences of the analyte, and wherein the second probe oligonucleotide comprises a capture probe binding domain; (b) coupling the first probe oligonucleotide and the second probe oligonucleotide, thereby generating a connected probe (e.g., a ligation product) comprising the capture probe binding domain; (c) contacting the biological sample with a reagent medium comprising a permeabilization agent and an agent for releasing the connected probe (e.g., a ligation product), thereby (i) permeabilizing the biological sample and (ii) releasing the connected probe (e.g., a ligation product) from the analyte; and (d) hybridizing the capture probe binding domain of the connected probe (e.g., a ligation product) to a capture domain of a capture probe, wherein the capture probe comprises: (i) a spatial barcode and (ii) a capture domain.

Also provided herein are methods for analyzing an analyte in a biological sample mounted on a first substrate including (a) hybridizing a first probe oligonucleotide and a second probe oligonucleotide to the analyte, wherein the first probe oligonucleotide and the second probe oligonucleotide each include a sequence that is substantially complementary to adjacent sequences of the analyte, and wherein the second probe oligonucleotide includes a capture probe binding domain; (b) coupling (e.g., ligating) the first probe oligonucleotide and the second probe oligonucleotide, thereby generating a connected probe (e.g., a ligation product) including the capture probe binding domain; (c) aligning the first substrate with a second substrate including an array, such that at least a portion of the biological sample is aligned with at least a portion of the array, wherein the array includes a plurality of capture probes, wherein a capture probe of the plurality of capture probes includes: (i) a spatial barcode and (ii) a capture domain; (d) when the biological sample is aligned with at least a portion of the array, (i) releasing the connected probe (e.g., a ligation product) from the analyte and (ii) passively or actively migrating the connected probe (e.g., a ligation product) from the biological sample to the array; and (e) hybridizing the capture probe binding domain of the connected probe (e.g., a ligation product) to the capture domain.

In some embodiments, the process of transferring the connected probe (e.g., a ligation product) from the first substrate to the second substrate is referred to as a “sandwich” process. The sandwich process is described in PCT Patent Application Publication No. WO 2020/123320, which is incorporated by reference in its entirety. Described herein are methods in which an array with capture probes located on a substrate and a biological sample located on a different substrate, are contacted such that the array is in contact with the biological sample (e.g., the substrates are sandwiched together). In some embodiments, the array and the biological sample can be contacted (e.g., sandwiched), without the aid of a substrate holder. In some embodiments, the array and biological sample substrates can be placed in a substrate holder (e.g., an array alignment device) designed to align the biological sample and the array. For example, the substrate holder can have placeholders for two substrates. In some embodiments, an array including capture probes can be positioned on one side of the substrate holder (e.g., in a first substrate placeholder). In some embodiments, a biological sample can be placed on the adjacent side of the substrate holder in a second placeholder. In some embodiments, a hinge can be located between the two substrate placeholders that allows the substrate holder to close, e.g., make a sandwich between the two substrate placeholders. In some embodiments, when the substrate holder is closed the biological sample and the array with capture probes are contacted with one another under conditions sufficient to allow analytes present in the biological sample to interact with the capture probes of the array. For example, dried permeabilization reagents can be placed on the biological sample and rehydrated. A permeabilization solution can be flowed through the substrate holder to permeabilize the biological sample and allow analytes in the biological sample to interact with the capture probes. Additionally, the temperature of the substrates or permeabilization solution can be used to initiate or control the rate of permeabilization. For example, the substrate including the array, the substrate including the biological sample, or both substrates can be held at a low temperature to slow diffusion and permeabilization efficiency. Once sandwiched, in some embodiments, the substrates can be heated to initiate permeabilization and/or increase diffusion efficiency. Transcripts that are released from the permeabilized tissue can diffuse to the array and be captured by the capture probes. The sandwich can be opened, and cDNA synthesis can be performed on the array.

In some embodiments, the methods as disclosed herein include hybridizing of one or more probe oligonucleotide probe pairs (e.g., RTL probes) to adjacent or nearby sequences of a target analyte (e.g., RNA; e.g., mRNA) of interest. In some embodiments, the probe oligonucleotide pairs include sequences that are complementary or substantially complementary to an analyte. For example, in some embodiments, each probe oligonucleotide includes a sequence that is complementary or substantially complementary to an mRNA of interest (e.g., to a portion of the sequence of an mRNA of interest). In some embodiments, each target analyte includes a first target region and a second target region. In some embodiments, the methods include providing a plurality of first probe oligonucleotides and a plurality of second probe oligonucleotides, wherein a pair of probe oligonucleotides for a target analyte comprises both a first and second probe oligonucleotide. In some embodiments, a first probe oligonucleotide hybridizes to a first target region of the analyte, and the second probe oligonucleotide hybridizes to a second, adjacent or nearly adjacent target region of the analyte.

In some instances, the probe oligonucleotides are DNA molecules. In some instances, the first probe oligonucleotide is a DNA molecule. In some instances, the second probe oligonucleotide is a DNA molecule. In some instances, the first probe oligonucleotide comprises at least two ribonucleic acid bases at the 3′ end. In some instances, the second probe oligonucleotide comprises a phosphorylated nucleotide at the 5′ end.

RTL probes can be designed using methods known in the art. In some instances, probe pairs are designed to cover an entire transcriptome of a species (e.g., a mouse or a human). In some instances, RTL probes are designed to cover a subset of a transcriptome (e.g., a mouse or a human). In some instances, the methods disclosed herein utilize about 500, about 1000, about 2000, about 3000, about 4000, about 5000, about 6000, about 7000, about 8000, about 9000, about 10,000, about 15,000, about 20,000, or more probe pairs.

In some embodiments, one of the probe oligonucleotides of the pair of probe oligonucleotides for RTL includes a poly(A) sequence or a complement thereof. In some instances, the poly(A) sequence or a complement thereof is on the 5′ end of one of the probe oligonucleotides. In some instances, the poly(A) sequence or a complement thereof is on the 3′ end of one of the probe oligonucleotides. In some embodiments, one probe oligonucleotide of the pair of probe oligonucleotides for RTL includes a degenerate or UMI sequence. In some embodiments, the UMI sequence is specific to a particular target or set of targets. In some instances, the UMI sequence or a complement thereof is on the 5′ end of one of the probe oligonucleotides. In some instances, the UMI sequence or a complement thereof is on the 3′ end of one of the probe oligonucleotides.

In some instances, the first and second target regions of an analyte are directly adjacent to one another. In some embodiments, the complementary sequences to which the first probe oligonucleotide and the second probe oligonucleotide hybridize are 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, or about 150 nucleotides away from each other. Gaps between the probe oligonucleotides may first be filled prior to coupling (e.g., ligation), using, for example, dNTPs in combination with a polymerase such as polymerase mu, DNA polymerase, RNA polymerase, reverse transcriptase, VENT polymerase, Taq polymerase, and/or any combinations, derivatives, and variants (e.g., engineered mutants) thereof. In some embodiments, when the first and second probe oligonucleotides are separated from each other by one or more nucleotides, deoxyribonucleotides are used to extend and couple (e.g., ligate) the first and second probe oligonucleotides.

In some instances, the first probe oligonucleotide and the second probe oligonucleotide hybridize to an analyte on the same transcript. In some instances, the first probe oligonucleotide and the second probe oligonucleotide hybridize to an analyte on the same exon. In some instances, the first probe oligonucleotide and the second probe oligonucleotide hybridize to an analyte on different exons. In some instances, the first probe oligonucleotide and the second probe oligonucleotide hybridize to an analyte that is the result of a translocation event (e.g., in the setting of cancer). The methods provided herein make it possible to identify alternative splicing events, translocation events, and mutations that change the hybridization rate of one or both probe oligonucleotides (e.g., single nucleotide polymorphisms, insertions, deletions, point mutations).

In some embodiments, the first and/or second probe as disclosed herein includes at least two ribonucleic acid bases at the 3′ end; a functional sequence; a phosphorylated nucleotide at the 5′ end; and/or a capture probe binding domain. In some embodiments, the functional sequence is a primer sequence.

The “capture probe binding domain” is a sequence that is complementary to a particular capture domain present in a capture probe. In some embodiments, the capture probe binding domain includes a poly(A) sequence. In some embodiments, the capture probe binding domain includes a poly-uridine sequence, a poly-thymidine sequence, or a combination thereof. In some embodiments, the capture probe binding domain includes a random sequence (e.g., a random hexamer or octamer). In some embodiments, the capture probe binding domain is complementary to a capture domain in a capture probe that detects a particular target(s) of interest. In some embodiments, a capture probe binding domain blocking moiety that interacts with the capture probe binding domain is provided. In some embodiments, a capture probe binding domain blocking moiety includes a sequence that is complementary or substantially complementary to a capture probe binding domain. In some embodiments, a capture probe binding domain blocking moiety prevents the capture probe binding domain from binding the capture probe when present. In some embodiments, a capture probe binding domain blocking moiety is removed prior to binding the capture probe binding domain (e.g., present in a connected probe (e.g., a ligation product)) to a capture probe. In some embodiments, a capture probe binding domain blocking moiety comprises a poly-uridine sequence, a poly-thymidine sequence, or a combination thereof.

Hybridization of the probe oligonucleotides to the target analyte can occur at a target having a sequence that is 100% complementary to the probe oligonucleotide(s). In some embodiments, hybridization can occur at a target having a sequence that is at least (e.g. at least about) 80%, at least (e.g. at least about) 85%, at least (e.g. at least about) 90%, at least (e.g. at least about) 95%, at least (e.g. at least about) 96%, at least (e.g. at least about) 97%, at least (e.g. at least about) 98%, or at least (e.g. at least about) 99% complementary to the probe oligonucleotide(s). After hybridization, in some embodiments, the first probe oligonucleotide is extended. After hybridization, in some embodiments, the second probe oligonucleotide is extended. For example, in some instances a first probe oligonucleotide hybridizes to a target sequence upstream for a second oligonucleotide probe, whereas in other instances a first probe oligonucleotide hybridizes to a target sequence downstream of a second probe oligonucleotide.

In some embodiments, methods disclosed herein include a wash step after hybridizing the first and the second probe oligonucleotides. The wash step removes any unbound oligonucleotides and can be performed using any technique known in the art. In some embodiments, a pre-hybridization buffer is used to wash the sample. In some embodiments, a phosphate buffer is used. In some embodiments, multiple wash steps are performed to remove unbound oligonucleotides. For example, it is advantageous to decrease the amount of unhybridized probes present in a biological sample as they may interfere with downstream applications and methods.

In some embodiments, after hybridization of probe oligonucleotides (e.g., first and the second probe oligonucleotides) to the target analyte, the probe oligonucleotides (e.g., the first probe oligonucleotide and the second probe oligonucleotide) are coupled (e.g., ligated) together, creating a single connected probe (e.g., a ligation product) that is complementary to the target analyte. Ligation can be performed enzymatically or chemically, as described herein. For example, the first and second probe oligonucleotides are hybridized to the first and second target regions of the analyte, and the probe oligonucleotides are subjected to a nucleic acid reaction to ligate them together. For example, the probes may be subjected to an enzymatic ligation reaction using a ligase (e.g., T4 RNA ligase (Rnl2), a SplintR ligase, or a T4 DNA ligase). See, e.g., Zhang L., et al.; Archaeal RNA ligase from Thermoccocus kodakarensis for template dependent ligation RNA Biol. 2017; 14(1): 36-44 for a description of KOD ligase. A skilled artisan will understand that various reagents, buffers, cofactors, etc. may be included in a ligation reaction depending on the ligase being used.

In some embodiments, the first probe oligonucleotide and the second probe oligonucleotides are on a contiguous nucleic acid sequence. In some embodiments, the first probe oligonucleotide is on the 3′ end of the contiguous nucleic acid sequence. In some embodiments, the first probe oligonucleotide is on the 5′ end of the contiguous nucleic acid sequence. In some embodiments, the second probe oligonucleotide is on the 3′ end of the contiguous nucleic acid sequence. In some embodiments, the second probe oligonucleotide is on the 5′ end of the contiguous nucleic acid sequence.

In some embodiments, the method further includes hybridizing a third probe oligonucleotide to the first probe oligonucleotide and the second probe oligonucleotide such that the first probe oligonucleotide and the second probe oligonucleotide abut each other. In some embodiments, the third probe oligonucleotide comprises a sequence that is at least 80%, at least 85%, at least 90%, at least 95%, at least 96%, at least 97%, at least 98%, or at least 99% complementary to a portion of the first probe oligonucleotide that hybridizes to the third probe oligonucleotide. In some embodiments, the third probe oligonucleotide comprises a sequence that is 100% complementary to a portion of the first probe oligonucleotide that hybridizes to the third probe oligonucleotide. In some embodiments, the third probe oligonucleotide comprises a sequence that is at least 80%, at least 85%, at least 90%, at least 95%, at least 96%, at least 97%, at least 98%, or at least 99% complementary to a portion of the second probe oligonucleotide that hybridizes to the third probe oligonucleotide. In some embodiments, the third probe oligonucleotide comprises a sequence that is 100% complementary to a portion of the second probe oligonucleotide that hybridizes to the third probe oligonucleotide.

In some embodiments, a method for identifying a location of an analyte in a biological sample exposed to different permeabilization conditions includes (a) contacting the biological sample with a substrate, wherein the substrate comprises a plurality of capture probes, wherein a capture probe of the plurality of capture probes comprises a capture domain; (b) contacting the biological sample with a first probe oligonucleotide and a second probe oligonucleotide, wherein the first probe oligonucleotide and the second probe oligonucleotide are substantially complementary to adjacent sequences of the analyte, and wherein the second probe oligonucleotide comprises a capture probe-binding domain that is capable of binding to a capture domain of the capture probe; (c) hybridizing the first probe oligonucleotide and the second probe oligonucleotide to adjacent sequences of the analyte; (d) coupling (e.g., ligating) the first probe oligonucleotide and the second probe oligonucleotide, thereby creating a connected probe (e.g., a connected probe (e.g., a ligation product)) that is substantially complementary to the analyte; (e) releasing the connected probe (e.g., a ligation product) from the analyte; (f) hybridizing the capture probe-binding domain of the connected probe (e.g., a ligation product) to the hybridization domain of the capture probe; (g) hybridizing a padlock oligonucleotide to the connected probe (e.g., a ligation product) bound to the capture domain (e.g., such that the padlock oligonucleotide is circularized), wherein the padlock oligonucleotide comprises: (i) a first sequence that is substantially complementary to a first portion of the connected probe (e.g., a ligation product), (ii) a backbone sequence, and (iii) a second sequence that is substantially complementary to a second portion of the connected probe (e.g., a ligation product); and (i) ligating and amplifying the circularized padlock oligonucleotide (e.g., using rolling circle amplification using the circularized padlock oligonucleotide as a template), thereby creating an amplified circularized padlock oligonucleotide, and using the amplified circularized padlock oligonucleotide to identify the location of the analyte in the biological sample.

In some embodiments, the method further includes amplifying the connected probe (e.g., a ligation product) prior to the releasing step. In some embodiments, the entire connected probe (e.g., a ligation product) is amplified. In some embodiments, only part of the connected probe (e.g., a ligation product) is amplified. In some embodiments, amplification is isothermal. In some embodiments, amplification is not isothermal. Amplification can be performed using any of the methods described herein such as, but not limited to, a strand-displacement amplification reaction, a rolling circle amplification reaction, a ligase chain reaction, a transcription-mediated amplification reaction, an isothermal amplification reaction, and/or a 10 loop-mediated amplification reaction. In some embodiments, amplifying the connected probe (e.g., a ligation product) creates an amplified connected probe (e.g., a ligation product) that includes (i) all or part of sequence of the connected probe (e.g., a ligation product) specifically bound to the capture domain, or a complement thereof, and (ii) all or a part of the sequence of the spatial barcode, or a complement thereof.

In some embodiments, the method further includes determining (i) all or a part of the sequence of the connected probe (e.g., a ligation product), or a complement thereof, and (ii) all or a part of the sequence of the spatial barcode, or a complement thereof. In some embodiments, the method further includes using the determined sequence of (i) and (ii) to determine the location and abundance of the analyte in the biological sample.

In some embodiments, after coupling (e.g., ligation) of the first and second probe oligonucleotides to create a ligation product, the connected probe (e.g., a ligation product) is released from the analyte. To release the connected probe (e.g., a ligation product), an endoribonuclease (e.g., RNase A, RNase C, RNase H, or RNase I) is used. An endoribonuclease such as RNase H specifically cleaves RNA in RNA:DNA hybrids. In some embodiments, the connected probe (e.g., a ligation product) is released enzymatically. In some embodiments, an endoribonuclease is used to release the probe from the analyte. In some embodiments, the endoribonuclease is one or more of RNase H. In some embodiments, the RNase H is RNase H1 or RNase H2.

In some embodiments, the releasing of the connected probe (e.g., a ligation product) includes contacting the biological sample with a reagent medium comprising a permeabilization agent and an agent for releasing the connected probe (e.g., a ligation product), thereby permeabilizing the biological sample and releasing the connected probe (e.g., a ligation product) from the analyte. In some embodiments, the agent for releasing the connected probe (e.g., a ligation product) comprises a nuclease. In some embodiments, the nuclease is an endonuclease. In some embodiments, the nuclease is an exonuclease. In some embodiments, the nuclease includes an RNase. In some embodiments, the RNase is selected from RNase A, RNase C, RNase H, or RNase I.

In some embodiments, the reagent medium comprises polyethylene glycol (PEG). In some embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, 15K, or 16K. In some embodiments, the PEG is present at a concentration from about 2% to 25%, from about 4% to about 23%, from about 6% to about 21%, or from about 8% to about 20% (v/v).

In some embodiments, the reagent medium includes a wetting agent.

In some instances, after creation of the connected probe (e.g., a ligation product), the methods disclosed herein include simultaneous treatment of the biological sample with a permeabilization agent such as proteinase K (to permeabilize the biological sample) and a releasing agent such as an endonuclease such as RNase H (to release the connected probe (e.g., a ligation product) from the analyte). In some instances, the permeabilization step and releasing step occur at the same time. In some instances, the permeabilization step occurs before the releasing step. In some embodiments, the permeabilization agent comprises a protease. In some embodiments, the protease is selected from trypsin, pepsin, elastase, or Proteinase K. In some embodiments, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin.

In some embodiments, the reagent medium further includes a detergent. In some embodiments, the detergent is selected from sodium dodecyl sulfate (SDS), sarkosyl, saponin, Triton X-100™, or Tween-20™. In some embodiments, the reagent medium includes less than 5 w/v % of a detergent selected from sodium dodecyl sulfate (SDS) and sarkosyl. In some embodiments, the reagent medium includes as least 5% w/v % of a detergent selected from SDS and sarkosyl. In some embodiments, the reagent medium does not include SDS or sarkosyl.

In some embodiments, the biological sample and the array are contacted with the reagent medium for about 1 to about 60 minutes (e.g., about 1 to about 55 minutes, about 1 to about 50 minutes, about 1 to about 45 minutes, about 1 to about 40 minutes, about 1 to about 35 minutes, about 1 to about 30 minutes, about 1 to about 25 minutes, about 1 to about 20 minutes, about 1 to about 15 minutes, about 1 to about 10 minutes, about 1 to about 5 minutes, about 5 to about 60 minutes, about 5 to about 55 minutes, about 5 to about 50 minutes, about 5 to about 45 minutes, about 5 to about 40 minutes, about 5 to about 35 minutes, about 5 to about 30 minutes, about 5 to about 25 minutes, about 5 to about 20 minutes, about 5 to about 15 minutes, about 5 to about 10 minutes, about 10 to about 60 minutes, about 10 to about 55 minutes, about 10 to about 50 minutes, about 10 to about 45 minutes, about 10 to about 40 minutes, about 10 to about 35 minutes, about 10 to about 30 minutes, about 10 to about 25 minutes, about 10 to about 20 minutes, about 10 to about 15 minutes, about 15 to about 60 minutes, about 15 to about 55 minutes, about 15 to about 50 minutes, about 15 to about 45 minutes, about 15 to about 40 minutes, about 15 to about 35 minutes, about 15 to about 30 minutes, about 15 to about 25 minutes, about 15 to about 20 minutes, about 20 to about 60 minutes, about 20 to about 55 minutes, about 20 to about 50 minutes, about 20 to about 45 minutes, about 20 to about 40 minutes, about 20 to about 35 minutes, about 20 to about 30 minutes, about 20 to about 25 minutes, about 25 to about 60 minutes, about 25 to about 55 minutes, about 25 to about 50 minutes, about 25 to about 45 minutes, about 25 to about 40 minutes, about 25 to about 35 minutes, about 25 to about 30 minutes, about 30 to about 60 minutes, about 30 to about 55 minutes, about 30 to about 50 minutes, about 30 to about 45 minutes, about 30 to about 40 minutes, about 30 to about 35 minutes, about 35 to about 60 minutes, about 35 to about 55 minutes, about 35 to about 50 minutes, about 35 to about 45 minutes, about 35 to about 40 minutes, about 40 to about 60 minutes, about 40 to about 55 minutes, about 40 to about 50 minutes, about 40 to about 45 minutes, about 45 to about 60 minutes, about 45 to about 55 minutes, about 45 to about 50 minutes, about 50 to about 60 minutes, about 50 to about 55 minutes, or about 55 to about 60 minutes). In some embodiments, the biological sample and the array are contacted with the reagent medium for about 30 minutes.

In some embodiments, the connected probe (e.g., a ligation product) includes a capture probe binding domain, which can hybridize to a capture probe (e.g., a capture probe immobilized, directly or indirectly, on a substrate). In some embodiments, the capture probe includes a spatial barcode and the capture domain. In some embodiments, the capture probe binding domain of the connected probe (e.g., a ligation product) specifically binds to the capture domain of the capture probe.

In some embodiments, methods provided herein include mounting a biological sample on a first substrate, then aligning the first substrate with a second substrate including an array, such that at least a portion of the biological sample is aligned with at least a portion of the array, wherein the array includes a plurality of capture probes. After hybridization of the connected probe (e.g., a ligation product) to the capture probe, downstream methods as disclosed herein can be performed.

In some embodiments, at least 50% of connected probes (e.g., a ligation products) released from the portion of the biological sample aligned with the portion of the array are captured by capture probes of the portion of the array. In some embodiments, at least 50%, at least 55%, at least 60%, at least 65%, at least 70%, at least 75%, at least 85%, at least 90%, at least 95%, at least 96%, at least 97%, at least 98%, at least 99%, or 100% of connected probe (e.g., a ligation products) are detected in spots directly under the biological sample.

In some embodiments, the capture probe includes a poly(T) sequence. In some embodiments, capture probe includes a sequence specific to the analyte. In some embodiments, the capture probe includes a functional domain. In some embodiments, the capture probe further includes one or more functional domains, a unique molecular identifier (UMI), a cleavage domain, and combinations thereof. In some embodiments, the capture probe binding domain includes a poly(A) sequence. In some embodiments, the capture probe binding domain includes a sequence complementary to a capture domain of a capture probe that detects a target analyte of interest. In some embodiments, the analyte is RNA. In some embodiments, the analyte is mRNA.

In some embodiments, the connected probe (e.g., a ligation product) (e.g., the analyte derived molecule) includes a capture probe binding domain, which can hybridize to a capture probe (e.g., a capture probe immobilized, directly or indirectly, on a substrate). Methods provided herein include contacting a biological sample with a substrate, wherein the capture probe is affixed to the substrate (e.g., immobilized to the substrate, directly or indirectly). After hybridization of the connected probe (e.g., a ligation product) to the capture probe, downstream methods as disclosed herein (e.g., sequencing, in situ analysis such as RCA) can be performed.

In some embodiments, the method further includes analyzing a different analyte in the biological sample. In some embodiments, the analysis of the different analyte includes (a) further contacting the biological sample with a plurality of analyte capture agents, wherein an analyte capture agent of the plurality of analyte capture agents includes an analyte binding moiety and a capture agent barcode domain, wherein the analyte binding moiety specifically binds to the different analyte, and wherein the capture agent barcode domain includes an analyte binding moiety barcode and an capture handle sequence that is complementary to a capture domain of a capture probe; and (b) hybridizing the analyte capture sequence to the capture domain.

In some examples, a fixed tissue sample mounted on a first substrate (e.g., a slide-mounted tissue sample) is decrosslinked, followed by hybridization of probe pairs to nucleic acid analyte analytes. Also, a first and second probe of a probe pair can optionally be connected, e.g., ligated. The sample is optionally washed (e.g., with a buffer), prior to incubation with an analyte capture agent (e.g., an antibody) that specifically binds a different analyte, e.g., a protein analyte. The analyte capture agent comprises a capture agent barcode domain. In some embodiments, the analyte capture agent is an antibody with an oligonucleotide tag, the oligonucleotide tag comprising a capture agent barcode domain. In some embodiments, the connected probes (e.g., the ligation products) and antibody oligonucleotide tags are released from the tissue under sandwich conditions as described herein. For the sandwich conditions, the tissue-mounted slide can be aligned with an array and permeabilized with a reagent medium in the sandwich configuration as described herein. In some embodiments, the reagent medium comprises RNase and a permeabilization agent (e.g., Proteinase K). Permeabilization releases the connected probe (e.g., a ligation product) and capture agent barcode domain, for capture onto a second substrate comprising an array with a plurality of capture probes. After capture of the connected probe and capture agent barcode domain, the tissue slide can be removed (e.g., the sandwich can be “opened” or “broken”).

In some embodiments, following opening of the sandwich, the capture probes can be extended, sequencing libraries can be prepared and sequenced, and the results can be analyzed computationally.

In some embodiments, the method further includes determining (i) all or part of the sequence of the capture agent barcode domain; and (ii) all or a part of the sequence of the spatial barcode, or a complement thereof. In some embodiments, the method further includes using the determined sequence of (i), and (ii) to analyze the different analyte in the biological sample. In some embodiments, the releasing step further releases the capture agent barcode domain from the different analyte. In some embodiments, the different analyte is a protein analyte. In some embodiments, the protein analyte is an extracellular protein. In some embodiments, the protein analyte is an intracellular protein.

### (C) Capturing Analytes for Spatial Detection Using Analyte Capture Agents

In some embodiments, the methods, compositions, devices, and systems provided herein utilize analyte capture agents for spatial detection. An “analyte capture agent” refers to a molecule that interacts with a target analyte (e.g., a protein) and with a capture probe. Such analyte capture agents can be used to identify the analyte. In some embodiments, the analyte capture agent can include an analyte binding moiety and a capture agent barcode domain. In some embodiments, the analyte capture agent includes a linker. In some embodiments, the linker is a cleavable linker. In some embodiments, the cleavable linker is a photo-cleavable linker, a UV-cleavable linker, or an enzyme cleavable linker.

An analyte binding moiety is a molecule capable of binding to a specific analyte. In some embodiments, the analyte binding moiety comprises an antibody or antibody fragment. In some embodiments, the analyte binding moiety comprises a polypeptide and/or an aptamer. In some embodiments, the analyte is a protein (e.g., a protein on a surface of a cell or an intracellular protein).

A capture agent barcode domain can include a capture handle sequence which can hybridize to at least a portion or an entirety of a capture domain of a capture probe. In some embodiments, the capture handle sequence is complementary to a portion or entirety of a capture domain of a capture probe. In some embodiments, the capture handle sequence includes a poly (A) tail. In some embodiments, the capture handle sequence includes a sequence capable of binding a poly (T) domain. In some embodiments, the capture agent barcode domain comprises an analyte binding moiety barcode and a capture handle sequence. The analyte binding moiety barcode refers to a barcode that is associated with or otherwise identifies the analyte binding moiety, and the capture handle sequence can hybridize to a capture probe. In some embodiments, the capture handle sequence specifically binds to the capture domain of the capture probe. Other embodiments of an analyte capture agent useful in spatial analyte detection are described herein.

Provided herein are methods for analyzing an analyte in a biological sample including (a) contacting the biological sample with a plurality of analyte capture agents, wherein an analyte capture agent of the plurality of analyte capture agents includes an analyte binding moiety and a capture agent barcode domain, wherein the analyte binding moiety specifically binds to the analyte, and wherein the capture agent barcode domain includes an analyte binding moiety barcode and an capture handle sequence; (b) contacting the biological sample with a reagent medium including an agent for releasing the capture agent barcode domain from the analyte binding moiety, thereby releasing the capture agent barcode domain from the analyte binding moiety; and (c) hybridizing the capture handle sequence to a capture domain of a capture probe, wherein the capture probe includes (i) a spatial barcode and (ii) a capture domain.

Also provided herein are methods for analyzing an analyte in a biological sample mounted on a first substrate including (a) contacting the biological sample with a plurality of analyte capture agents, wherein an analyte capture agent of the plurality of analyte capture agents includes an analyte binding moiety and a capture agent barcode domain, wherein the analyte binding moiety specifically binds to the analyte, and wherein the capture agent barcode domain includes an analyte binding moiety barcode and an capture handle sequence; (b) aligning the first substrate with a second substrate comprising an array, such that at least a portion of the biological sample is aligned with at least a portion of the array, wherein the array includes a plurality of capture probes, wherein a capture probe of the plurality of capture probes includes (i) a spatial barcode and (ii) a capture domain; (c) when the biological sample is aligned with at least a portion of the array, (i) releasing the capture agent barcode domain from the analyte and (ii) passively or actively migrating the capture agent barcode domain from the biological sample to the array; and (d) coupling the capture handle sequence to the capture domain.

Also provided herein are methods for analyzing an analyte in a biological sample mounted on a first substrate including (a) contacting the biological sample with a plurality of analyte capture agents, wherein an analyte capture agent of the plurality of analyte capture agents includes an analyte binding moiety and a capture agent barcode domain, wherein the analyte binding moiety specifically binds to the analyte, and wherein the capture agent barcode domain includes an analyte binding moiety barcode and an capture handle sequence; (b) aligning the first substrate with a second substrate comprising an array, such that at least a portion of the biological sample is aligned with at least a portion of the array, wherein the array includes a plurality of capture probes, wherein a capture probe of the plurality of capture probes includes (i) a spatial barcode and (ii) a capture domain; (c) when the biological sample is aligned with at least a portion of the array, (i) releasing the capture agent barcode domain from the analyte and (ii) passively or actively migrating the capture agent barcode domain from the biological sample to the array; and (d) hybridizing the capture handle sequence to the capture domain.

In some embodiments, the process of transferring the connected probe (e.g., a ligation product) from the first substrate to the second substrate is referred to as a “sandwich process”. The sandwich process is described above and in PCT Patent Application Publication No. WO 2020/123320, which is incorporated by reference in its entirety.

In some embodiments, the method further includes determining (i) all or a part of the capture agent barcode domain, or a complement thereof; and (ii) all or a part of the sequence of the spatial barcode, or a complement thereof. In some embodiments, the method further includes using the determined sequence of (i) and (ii) to determine the location and abundance of the analyte in the biological sample.

In some embodiments, an analyte capture agent is introduced to a biological sample, wherein the analyte binding moiety specifically binds to a target analyte, and then the biological sample can be treated to release the capture agent barcode domain from the biological sample. In some embodiments, the capture agent barcode domain can then migrate and bind to a capture domain of a capture probe, and the capture agent barcode domain can be extended to generate a spatial barcode complement at the end of the capture agent barcode domain. In some embodiments, the spatially-tagged capture agent barcode domain can be denatured from the capture probe, and analyzed using methods described herein.

In some embodiments, the releasing includes contacting the biological sample and the array with a reagent medium including a nuclease. In some embodiments, the nuclease includes an RNase. In some embodiments, the RNase is selected from RNase A, RNase C, RNase H, and RNase I. In some embodiments, the reagent medium further includes a permeabilization agent. In some embodiments, the releasing further includes simultaneously permeabilizing the biological sample and releasing the capture agent barcode domain from the analyte. In some embodiments, the permeabilization agent further includes a protease. In some embodiments, the protease is selected from trypsin, pepsin, elastase, or Proteinase K.

In some embodiments, the capture agent barcode domain is released from the analyte binding moiety by using a different stimulus that can include, but is not limited to, a proteinase (e.g., Proteinase K), an RNase, and UV light.

In some embodiments, the reagent medium further includes a detergent. In some embodiments, the detergent is selected from sodium dodecyl sulfate (SDS), sarkosyl, saponin, Triton X-100™, or Tween-20™. In some embodiments, the reagent medium includes less than 5 w/v % of a detergent selected from sodium dodecyl sulfate (SDS) and sarkosyl. In some embodiments, the reagent medium includes as least 5% w/v % of a detergent selected from SDS and sarkosyl. In some embodiments, the reagent medium does not include SDS or sarkosyl.

In some embodiments, the biological sample and the array are contacted with the reagent medium for about 1 to about 60 minutes (e.g., about 1 to about 55 minutes, about 1 to about 50 minutes, about 1 to about 45 minutes, about 1 to about 40 minutes, about 1 to about 35 minutes, about 1 to about 30 minutes, about 1 to about 25 minutes, about 1 to about 20 minutes, about 1 to about 15 minutes, about 1 to about 10 minutes, about 1 to about 5 minutes, about 5 to about 60 minutes, about 5 to about 55 minutes, about 5 to about 50 minutes, about 5 to about 45 minutes, about 5 to about 40 minutes, about 5 to about 35 minutes, about 5 to about 30 minutes, about 5 to about 25 minutes, about 5 to about 20 minutes, about 5 to about 15 minutes, about 5 to about 10 minutes, about 10 to about 60 minutes, about 10 to about 55 minutes, about 10 to about 50 minutes, about 10 to about 45 minutes, about 10 to about 40 minutes, about 10 to about 35 minutes, about 10 to about 30 minutes, about 10 to about 25 minutes, about 10 to about 20 minutes, about 10 to about 15 minutes, about 15 to about 60 minutes, about 15 to about 55 minutes, about 15 to about 50 minutes, about 15 to about 45 minutes, about 15 to about 40 minutes, about 15 to about 35 minutes, about 15 to about 30 minutes, about 15 to about 25 minutes, about 15 to about 20 minutes, about 20 to about 60 minutes, about 20 to about 55 minutes, about 20 to about 50 minutes, about 20 to about 45 minutes, about 20 to about 40 minutes, about 20 to about 35 minutes, about 20 to about 30 minutes, about 20 to about 25 minutes, about 25 to about 60 minutes, about 25 to about 55 minutes, about 25 to about 50 minutes, about 25 to about 45 minutes, about 25 to about 40 minutes, about 25 to about 35 minutes, about 25 to about 30 minutes, about 30 to about 60 minutes, about 30 to about 55 minutes, about 30 to about 50 minutes, about 30 to about 45 minutes, about 30 to about 40 minutes, about 30 to about 35 minutes, about 35 to about 60 minutes, about 35 to about 55 minutes, about 35 to about 50 minutes, about 35 to about 45 minutes, about 35 to about 40 minutes, about 40 to about 60 minutes, about 40 to about 55 minutes, about 40 to about 50 minutes, about 40 to about 45 minutes, about 45 to about 60 minutes, about 45 to about 55 minutes, about 45 to about 50 minutes, about 50 to about 60 minutes, about 50 to about 55 minutes, or about 55 to about 60 minutes). In some embodiments, the biological sample and the array are contacted with the reagent medium for about 30 minutes.

Also provided herein are methods further including analyzing a different analyte in the biological sample. In some embodiments, the analysis of the different analyte includes (a) hybridizing a first probe oligonucleotide and a second probe oligonucleotide to the different analyte, wherein the first probe oligonucleotide and the second probe oligonucleotide each comprise a sequence that is substantially complementary to adjacent sequences of the different analyte, and wherein the second probe oligonucleotide comprises a capture probe binding domain; (b) ligating the first probe oligonucleotide and the second probe oligonucleotide, thereby generating a connected probe (e.g., a ligation product) comprising the capture probe binding domain; and (c) hybridizing the capture probe binding domain of the connected probe (e.g., a ligation product) to the capture domain.

In some embodiments, the method further includes determining (i) all or part of the sequence of the connected probe (e.g., a ligation product), or a complement thereof, and (ii) all or a part of the sequence of the spatial barcode, or a complement thereof. In some embodiments, the method further includes using the determined sequence of (i), and (ii) to analyze the different analyte in the biological sample. In some embodiments, the releasing step further releases the connected probe (e.g., a ligation product) from the different analyte. In some embodiments, the different analyte is RNA. In some embodiments, the different analyte is mRNA.

In some embodiments, the capture probe comprises a poly(T) sequence. In some embodiments, the capture probe comprises a sequence complementary to the capture handle sequence. In some embodiments, the capture probe comprises a functional domain. In some embodiments, the capture probe further comprises one or more functional domains, a unique molecular identifier (UMI), a cleavage domain, and combinations thereof.

In some embodiments, the biological sample is a tissue sample. In some embodiments, the tissue sample is a tissue section. In some embodiments, the tissue sample is a fixed tissue sample. In some embodiments, the fixed tissue sample is a formalin fixed paraffin embedded (FFPE) tissue sample. In some embodiments, the FFPE tissue is deparaffinized and decrosslinked prior to step (a) of any one of the methods provided herein. In some embodiments, the fixed tissue sample is a formalin fixed paraffin embedded cell pellet. In some embodiments, the tissue sample is a fresh tissue sample or a frozen tissue sample. In some embodiments, the tissue sample is fixed and stained prior to step (a) of any one of the methods provided herein.

In some instances, RTL is performed between two oligonucleotides that each are affixed to an analyte binding moiety (i.e., a protein-binding moiety). Generally, the methods of RTL in this setting is as follows. In some embodiments, provided herein is a method of determining a location of at least one analyte in a biological sample including: (a) hybridizing a first analyte-binding moiety to a first analyte in the biological sample, wherein the first analyte-binding moiety is bound to a first oligonucleotide, wherein the first oligonucleotide comprises: (i) a functional sequence; (ii) a first barcode; and (iii) a first bridge sequence; (b) hybridizing a second analyte-binding moiety to a second analyte in the biological sample, wherein the second analyte-binding moiety is bound to a second oligonucleotide; wherein the second oligonucleotide comprises: (i) capture probe binding domain sequence, (ii) a second barcode; and (ii) a second bridge sequence; (c) contacting the biological sample with a third oligonucleotide; (d) hybridizing the third oligonucleotide to the first bridge sequence of the first oligonucleotide and second bridge sequence of the second oligonucleotide; (e) ligating the first oligonucleotide and the second oligonucleotide, creating a connected probe (e.g., a ligation product); (f) contacting the biological sample with a substrate, wherein a capture probe is affixed to the substrate, wherein the capture probe comprises a spatial barcode and the capture domain; and (g) allowing the capture probe binding domain sequence of the second oligonucleotide to specifically bind to the capture domain. In some instances, the connected probe (e.g., a ligation product) is cleaved from the analyte biding moieties.

In some instances, two analytes (e.g., two different proteins) in close proximity in a biological sample are detected by a first analyte-binding moiety and a second analyte-binding moiety, respectively. In some embodiments, a first analyte-binding moiety and/or the second analyte-binding moiety is an analyte capture agent (e.g., any of the exemplary analyte capture agents described herein). In some embodiments, the first analyte-binding moiety and/or the second analyte-binding moiety is a first protein. In some embodiments, the first analyte-binding moiety and/or the second analyte-binding moiety is an antibody. For example, the antibody can include, without limitation, a monoclonal antibody, recombinant antibody, synthetic antibody, a single domain antibody, a single-chain variable fragment (scFv), and or an antigen-binding fragment (Fab). In some embodiments, the first analyte-binding moiety binds to a cell surface analyte (e.g., any of the exemplary cell surface analytes described herein). In some embodiments, binding of the analyte is performed metabolically. In some embodiments, binding of the analyte is performed enzymatically. In some embodiments, the methods include a secondary antibody that binds to a primary antibody, enhancing its detection.

In some embodiments, the first analyte-binding moiety and the second analyte-binding moiety each bind to the same analyte. In some embodiments, the first analyte-binding moiety and/or second analyte-binding moiety each bind to a different analyte. For example, in some embodiments, the first analyte-binding moiety binds to a first polypeptide and the second analyte-binding moiety binds to a second polypeptide.

In some embodiments of any of the methods of determining a location of at least one analyte in a biological sample, a first and/or a second oligonucleotide are bound (e.g., conjugated or otherwise attached using any of the methods described herein) to a first analyte-binding moiety and/or a second analyte-binding moiety, respectively.

In some embodiments of any of the methods of determining a location of at least one analyte in a biological sample as described herein, a second oligonucleotide is bound (e.g., conjugated or otherwise attached using any of the methods described herein) to a second analyte-binding moiety. For example, the second oligonucleotide can be covalently linked to the second analyte-binding moiety. In some embodiments, the second oligonucleotide is bound to the second analyte-binding moiety via its 5′ end. In some embodiments, the second oligonucleotide includes a free 3′ end. In some embodiments the second oligonucleotide is bound to the second analyte-binding moiety via its 3′ end. In some embodiments, the second oligonucleotide includes a free 5′ end.

In some embodiments, the oligonucleotides are bound to the first and/or second analyte-binding moieties via a linker (e.g., any of the exemplary linkers described herein). In some embodiments, the linker is a cleavable linker. In some embodiment, the linker is a linker with photo-sensitive chemical bonds (e.g., photo-cleavable linkers). In some embodiments, the linker is a cleavable linker that can undergo induced dissociation.

In some embodiments, the oligonucleotides are bound (e.g., attached via any of the methods described herein) to an analyte-binding domain via a 5′ end.

In some embodiments, a barcode is used to identify the analyte-binding moiety to which it is bound. The barcode can be any of the exemplary barcodes described herein. In some embodiments, the first and/or second oligonucleotide include a capture probe binding domain sequence. For example, a capture probe binding domain sequence can be a poly(A) sequence when the capture domain sequence is a poly(T) sequence.

In some embodiments, a third oligonucleotide (e.g., a splint oligonucleotide) hybridizes to both the first and second oligonucleotides and enables ligation of the first oligonucleotide and the second oligonucleotide. In some embodiments, a ligase is used. In some aspects, the ligase includes a DNA ligase. In some aspects, the ligase includes a RNA ligase. In some aspects, the ligase includes T4 DNA ligase. In some embodiments, the ligase is a SplintR ligase.

### (D) Sandwich Processes

One or more analytes from the biological sample can be released from the biological sample and migrate to a substrate comprising an array of capture probes for attachment to the capture probes of the array. In some embodiments, the release and migration of the analytes to the substrate comprising the array of capture probes occurs in a manner that preserves the original spatial context of the analytes in the biological sample. In some embodiments, the biological sample is mounted on a first substrate and the substrate comprising the array of capture probes is a second substrate. In some embodiments, the alignment of the first substrate and the second substrate is facilitated by a sandwiching process. Accordingly, described herein are methods, compositions, devices, and systems for sandwiching together the first substrate as described herein with a second substrate having an array with capture probes.

FIG. 12 is an exemplary embodiment of a sample handling system 1200 (also can be referred to as “SHS” or a sandwiching system), as further described in later sections herein. The SHS 1200 can include one or more housings 1202, 1204 for handling a sample (e.g., a biological sample). The sample, e.g., biological sample or the tissue sample, can be fixed without chemical fixation (e.g., chemical fixatives). A “fixed” biological sample (e.g., tissue sample, tissue section) exhibits reduced biochemical reactions (e.g., degradation or decay), or increased stabilization of cellular structures and molecular components compared to an unfixed biological sample. For example, the biological sample can be fixed by freezing the biological sample on a sample substrate (e.g., first substrate 1214, slide, or other substrate). Fixation can be maintained by controlling the temperature of the first substrate 1214. In some embodiments, the fixation is performed after sectioning. In some examples, the first substrate 1214 can be kept at a threshold temperature (e.g., such that a biological sample disposed thereon stays frozen and/or fixed), outside of the SHS 1200. For example, the first substate 1214 can be in contact with a thermal element outside of the SHS 1200 that controls the temperature of the first substrate. The thermal element outside of the SHS 1200 can operate in a same or similar manner to the thermal element 1203 and/or the thermal element 1203b. In some embodiments, the biological sample can be flash-frozen onto the substrate, and the external thermal element can maintain fixation in the frozen section until loading of the substrate into the SHS 1200. The external thermal element can be an optional element. For example, in some embodiments the external thermal element is not used and the thermal element 1203 and/or 1203b maintain the sample fixation until release of analytes is needed.

In some embodiments, the thermal element 1203 and/or 1203b facilitate the freezing of or keeping frozen (and maintaining fixation) of a biological sample (e.g., tissue sample, tissue section) and targets within the biological sample. In some embodiments, a heating mode of the thermal element 1203 and/or 1203b can facilitate the thawing or release of a frozen tissue sample and targets within the sample. For example, the thermal element 1203 is configured to maintain a surface temperature of the first substrate 1214 between −20° C. and 5° C. (e.g., a cooling and/or freezing effect). The thermal element 1203 is configured to maintain a surface temperature of the first substrate 1214 around 0° C. (e.g., a cooling and/or freezing effect). The thermal element 1203 is configured to maintain a surface temperature of the first substrate 1214 between 10° C. and 40° C. (e.g., a heating and/or thawing effect). The thermal element 1203 is configured to, upon energizing the thermal element 1203 in a cooling mode, maintain a surface temperature of the first substrate 1214 between −20° C. and 5° C. The thermal element 1203 is configured to, upon energizing the thermal element 1203 in a heating mode, maintain a surface temperature of the first substrate 1214 between 10° C. and 40° C.

In some embodiments, the biological sample, e.g., the tissue sample is fixed, for example by freezing and without chemical fixatives. In some embodiments, the biological sample is not fixed in methanol, acetone, acetone-methanol, PFA, PAXgene. In some embodiments, the biological sample is not formalin-fixed and paraffin-embedded (FFPE). A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. Sample fixation by freezing obviates the disadvantages of the chemical fixation processes.

The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time can be needed for staining and imaging of different types of biological samples.

Referring back to FIG. 12, a perspective view of an example embodiment of the sample handling system (SHS) 100 is shown. The SHS 1200 can include an upper housing 1202 and a lower housing 1204. Some embodiments of the SHS 1200 also include a thermal element 1203. The upper housing 1202, the lower housing 1204, or both the upper housing 1202 and the lower housing 1204 can include the thermal element 1203. The thermal element 1203 is configured to control the temperature (e.g., heat and/or cool) of one or more sample substrates, sample slides, samples, or other substrates of that the sample handing system 1200 can interface with. In some embodiments, the thermal element 1203 is a cold plate, a cooling plate, or otherwise temperature controlled plate, slide, plane, or other surfaces that are temperature controlled (e.g., to cool and optionally to heat). The temperature control of the thermal element 1203 can facilitate transfer of temperature control from the thermal element 1203 to other surfaces that contact the thermal element (e.g., the first substrate 1214). The upper housing 1202 and the lower housing 1204 can be connected at an alignment mechanism 1208 (e.g., via a hinge) to facilitate actuation of the upper housing 1202 from an open configuration (that facilitates loading of one or more sample slides and/or a capture array slide) to a closed configuration (that facilitates alignment of the sample slides and capture array slide). The upper housing 1202 can be rotated open and/or rotated closed with respect to the lower housing 1204. In various embodiments, in the closed configuration, a sample support member 1240 (onto which sample substrates are positioned and retained) has a predetermined spacing from a base support member 1220. The alignment mechanism 1208 can be configured to position the upper housing 1202 in an open configuration (as shown in FIG. 12) or in a closed configuration by opening or closing (e.g., via rotation) the upper housing 1202 relative to the lower housing 1204 in a clamshell manner via the alignment mechanism 1208. In the closed configuration, the upper housing 1202 can be rotated into contact with the lower housing 1204 such that the upper housing 1202 rests atop the lower housing 1204, and the sample support member 1240 is positioned opposite the base support member 1220. In various embodiments, the lower housing 1204 has a well (e.g., an extruded cut forming a depressed area in the lower housing 1204) and a raised lip defining a perimeter around the well. In various embodiments, the base support member 1220 extends from a base of the well. In various embodiments, the base support member 1220 extends to a lesser height compared to a height of the lip (does not extend to the same height as the lip) so that, when the upper housing 1202 and the lower housing 1204 are in the closed configuration, a gap is formed between the one or more sample slides positioned on the upper support member(s) and a capture array slide positioned on the base support member 1220.

The SHS 1200 can be provided on a suitable working surface, such that the lower housing 1204 can contact the working surface via a plurality of feet 1206 configured to extend from a bottom surface of the lower housing 1204. In various embodiments, the plurality of feet 1206 are adjustable such that a height of each foot 1206 can be adjusted in the z-direction to stabilize the SHS 1200. In various embodiments, the plurality of feet 1206 have vibration dampening features. For example, the plurality of feet 1206 can include a vibration dampening material (e.g., rubber) or can include vibration dampening mechanisms (e.g., one or more springs, one or more dampeners, one or more motion actuators such as a piezoelectric actuator, and/or one or more motion controllers).

In various embodiments, the upper housing 1202 includes one or more first retaining mechanisms 1210 and the lower housing 1204 includes one or more second retaining mechanisms 1212. The first retaining mechanisms 1210 can be configured to retain one or more first substrates 1214 (e.g., sample substrates). In FIG. 12, the upper housing 1202 is configured to retain two sample substrates (e.g., within the first retaining mechanisms 1210), however the upper housing 1202 can be configured to retain more (e.g., 3, 4, 5, or more) or fewer (e.g., 1) sample substrates. As further shown in FIG. 12, the SHS 1200 includes a second retaining mechanism 1212 including a spring-loaded member 1213. In various embodiments, the second retaining mechanism 1212 is disposed on the lower housing 1204 and is configured to receive and secure a second substrate 1216 (e.g., a capture array substrate/slide) to the lower housing 1204. In various embodiments, the second retaining mechanism 1212 includes a spring-loaded member 1213 and a spring 1215 (or the like) configured within the spring-loaded member 1213 such that the spring-loaded member 1213 is rotationally pivoted toward or away from the second substrate 1216 via the spring 1215. The spring-loaded member 1213 can be configured to provide a force against the second substrate 1216, such that, when a substrate (e.g., a glass slide) is positioned against the second retaining mechanism 1212, the second substrate 1216 is retained within the base support member 1220. In some embodiments, the second substrate 1216 can include an array substrate as described herein.

In various embodiments, the first retaining mechanism 1210 includes at least one clip 1211 (e.g., each first retaining mechanism has a clip). In some embodiments, the first retaining mechanism 1210 includes a spring (or the like) configured to ensure the clip 1211 maintains contact with the first substrate 1214. In some embodiments, the clip 1211 is pre-biased such that the clip provides a force against the sample support member 1240 even without the first substrate 1214 positioned thereon.

In some embodiments, the upper housing 1202 includes the thermal element 1203. The thermal element 1203 is configured to control a temperature at the first substrate 1214. The thermal element 1203 can be positioned in, housed in, or otherwise mounted to the upper housing 1202. In the open configuration illustrated in FIG. 12, the thermal element 1203 can be positioned behind the first substrate 1214 such that the thermal element 1203 is positioned between the first substrate 1214 and the sample support member 1240. In some embodiments, the thermal element 1203 can be positioned behind the sample support member 1240. In other embodiments, the thermal element 1203 can surround the first substrate 1214 (e.g., extend around a perimeter of the first substrate 1214.

The thermal element 1203 is configured to control the climate (e.g., a microclimate) surrounding the first substrate 1214. In some embodiments, the thermal element 1203 can control the temperature of the first substrate 1214 by contacting the first substrate 1214 with a surface of the thermal element 1203. For example, the thermal element 1203 can control the temperature at one or more surfaces of the thermal element 1203, and the temperature difference (e.g., heating or cooling) can be transferred from the thermal element 1203 to the first substrate 1214 to control the temperature of the first substrate 1214.

In some embodiments, the thermal element 1203 can include one or more temperature regulating devices. For example, the thermal element 1203 can be a thermoelectric cooler (TEC). The thermoelectric cooler can utilize the Peltier effect to generate a flow of heat from one side of the thermal element to another side. The flow of heat from one side to the other side facilitates the transfer of heat from one side of the thermal element 1203 to the other side. As a thermoelectric cooler, the thermal element 1203 can control a temperature (e.g., heating and cooling) at the first substrate 1214. In some embodiments, a current supplied to the thermal element 1203 can be applied in a first direction to generate a cooling effect at the thermoelectric cooler. For example, a current can be supplied to the thermal element 1203 in a second direction (e.g., opposite the first direction) to generate a heating effect at the thermoelectric cooler. In some embodiments, the thermal element 1203 can include one or more chambers of liquid nitrogen. The liquid nitrogen can be contained within the thermal element 1203, and contact between the thermal element 1203 and the first substrate 1214 can transfer temperature (E.g., cooling) from the thermal element 1203 to the first substrate 1214. In some embodiments, the thermal element 1203 includes a cooling liquid that circulates within the thermal element 1203 to cool the first substrate 1214.

The thermal element 1203 can be configured to, upon energizing the thermal element 1203, control and maintain a surface temperature of the first substrate 1214. The thermal element 1203 can control the surface temperature of the first substrate 1214 at various temperatures that range from provide a cooling effect to providing a heating effect. In some embodiments, the cooling effect can facilitate the freezing of or keeping frozen of a tissue sample and targets within the sample. In some embodiments, the heating effect can facilitate the thawing or release of a frozen tissue sample and targets within the sample. For example, the thermal element 1203 is configured to maintain a surface temperature of the first substrate 1214 between −20° C. and 5° C. (e.g., a cooling and/or freezing effect). The thermal element 1203 is configured to maintain a surface temperature of the first substrate 1214 around 0° C. (e.g., a cooling and/or freezing effect). The thermal element 1203 is configured to maintain a surface temperature of the first substrate 1214 between 10° C. and 40° C. (e.g., a heating and/or thawing effect). The thermal element 1203 is configured to, upon energizing the thermal element 1203 in a cooling mode, maintain a surface temperature of the first substrate 1214 between −20° C. and 5° C. The thermal element 1203 is configured to, upon energizing the thermal element 1203 in a heating mode, maintain a surface temperature of the first substrate 1214 between 10° C. and 40° C.

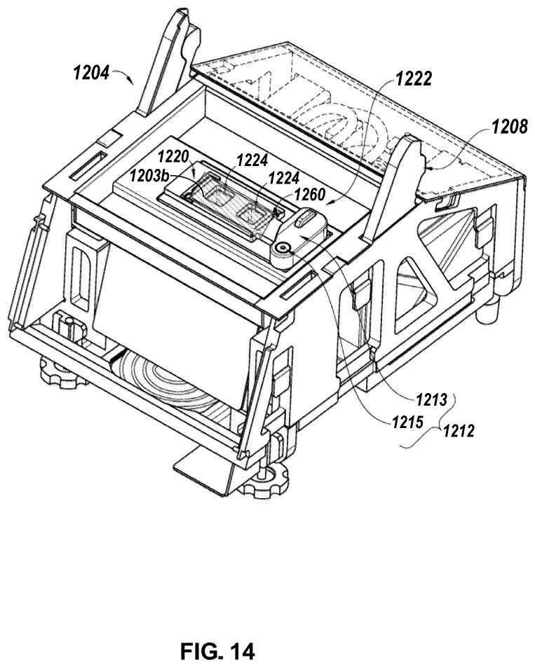

While the thermal element 1203 is shown at the upper housing 1202, the thermal element 1203 can be positioned at the lower housing 1204. In some embodiments, each of the upper housing 1202 and the lower housing 1204 includes a thermal element (e.g., SHS 1200 includes more than one thermal element 1203 and 1203b). The lower housing 1204 can include a similar thermal element to the thermal element 1203 (see e.g., optional thermal element 1203b illustrated in FIG. 14). The thermal element 1203b in the lower housing 1204 can control a temperature at a surface of the second substrate 1216. The thermal element in the lower housing 1204 can generate a climate and/or microclimate around the second substate 1216 in a same or similar manner to the thermal element 1203 described above.

In some embodiments, when the SHS 1200 is in an open position (as in FIG. 12), the first substrate 1214 and/or the second substrate 1216 can be loaded and positioned within the SHS 1200 such as within the upper housing 1202 and the lower housing 1204, respectively. As noted, the alignment mechanism 1208 can allow the upper housing 1202 to close over and onto the lower housing 1204 such that the one or more substrates on the sample support member 1240 are at a first distance from the one or more substrates on the base support member (and the one or more sample support members 1240 have a larger distance from the base support member). In various embodiments, when in the closed configuration, the upper housing 1202 contacts the lip (e.g., a portion of or an entirety of the lip) of the lower housing 1204. In various embodiments, the upper housing 1202 is configured to sandwich the first substrate 1214 and the second substrate 1216 by bringing the first substrate(s) and the second substrate(s) into close proximity and/or contact with one another.

In some aspects, after the upper housing 1202 closes over and onto the lower housing 1204, an adjustment mechanism of the sample handling system 1200 actuates portions of the upper housing 1202 and/or the lower housing 1204 (e.g., at least one sample support member 1240 and/or the base support member 1220) to adopt a sandwich configuration for a permeabilization step. For example, the adjustment mechanism involves bringing the at least one first substrate 1214 and the second substrate 1216 close to one other (e.g., via a linear actuator that actuates the sample support member 1240 towards the base support member 1220). In some aspects, the adjustment mechanism actuates the sample support member 1240 and/or the base support member 1220. The adjustment mechanism can be configured to control a speed, an angle, or the like of the sandwich configuration. In various embodiments, the adjustment mechanism includes a linear actuator. In various embodiments, the linear actuator includes a screw-driven actuator, a solenoid, a stepper motor, a hydraulic actuator, a pneumatic actuator, and/or a piezoelectric actuator.

In some embodiments, the thermal element 1203 (and/or a thermal element 1203b of the lower housing 1204) are controlled responsive to the position of the upper housing 1202 in relation to the lower housing 1204. For example, the thermal element 1203 can be operated in the cooling mode to keep a sample at the first substrate 1214 frozen. The thermal element 1203 can stay in the cooling mode to keep the sample frozen. In some embodiments, when the upper housing 1202 closes over and onto the lower housing 1204, the thermal element 1203 can automatically switch from the cooling mode to a heating mode. In some embodiments, when the upper housing 1202 closes over and onto the lower housing 1204, the thermal element 1203 can remain in the cooling mode (e.g., until a user inputs command instructions at the SHS 1200 to initiate a thawing process).

A tissue sample on the first substrate 1214 is secured within the upper housing 1202 (e.g., via the first retaining mechanism 1210) prior to closing the upper housing 1202 onto the lower housing 1204. A desired region of interest of the sample is aligned with a capture array of the second substrate 1216, e.g., when the first and the second substrates are aligned in the sandwich configuration. Such alignment can be accomplished manually (e.g., by a user) or automatically (e.g., via an automated mechanism). In some embodiments, during the alignment process, the thermal element 1203 and/or the thermal element 1203b of the lower housing 1204 can be active and controlling a temperature of the first substrate 1214 and/or the second substrate 1216. In various embodiments, before alignment, one or more spacers are applied to the first substrate 1214 and/or the second substrate 1216 to maintain a minimum spacing between the first substrate 1214 and the second substrate 1216 during sandwiching.

In some embodiments, a reagent (e.g., a permeabilization solution and/or an analyte transfer medium) is applied to the first substrate 1214 and/or the second substrate 1216. For example, one or more drops of a reagent is dispensed on one or more spacer (e.g., one drop for each capture area) that is adhered to the capture array slide. The upper housing 1202 can then close over the lower housing 1204 and form the sandwich configuration, thereby contacting the sample substrate(s) and capture area with the reagent (and providing a liquid medium between the samples and the capture areas). The thermal element 1203 can change from a cooling mode (e.g., maintaining a frozen state of the sample) to a heating mode or an off mode (e.g., thawing from the frozen state) to release the analytes. Analytes (e.g., mRNA transcripts, proteins, etc.) can migrate from the permeabilized sample on the first substrate 1214 captured by capture probes on the second substrate 1216 to be processed for spatial analysis.

In some embodiments, after the upper housing is in the closed configuration, an image capture device can capture one or more images of the overlap area between the tissue sample and the capture probes. If more than one first substrates 1214 and/or second substrates 1216 are present within the SHS 1200, the image capture device can be configured to capture one or more images of one or more overlap areas (for example, the SHS 1200 can have two or more cameras, and each camera is directed at a unique capture area on the second substrate). In some embodiments, particularly where the thermal element 1203 covers the area between the first substrate 1214 and upper housing 1202, the thermal element 1203 can be transparent. The SHS 1200 can include one or more image capture devices that can be configured to capture one or more images of one or more overlap areas through the thermal element 1203. While the thermal element 1203 can be transparent to facilitate image capture through the thermal element 1203, some embodiments include a thermal element 1203 that is not transparent. For example, the thermal element 1203 can be positioned at the upper housing 1202 in a manner that does not obstruct the image capture devices while controlling the temperature of the first substrate 1214. Non-limiting examples of such configurations include a thermal element 1203 that surrounds a perimeter of the first substrate 1214, or a thermal element 1203 that extends across the areas of the first substrate 1214 that do not obstruct the image capture device(s).

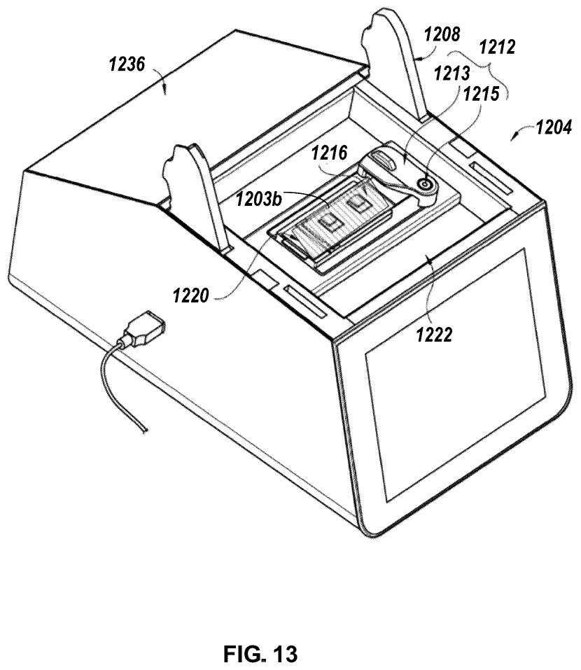

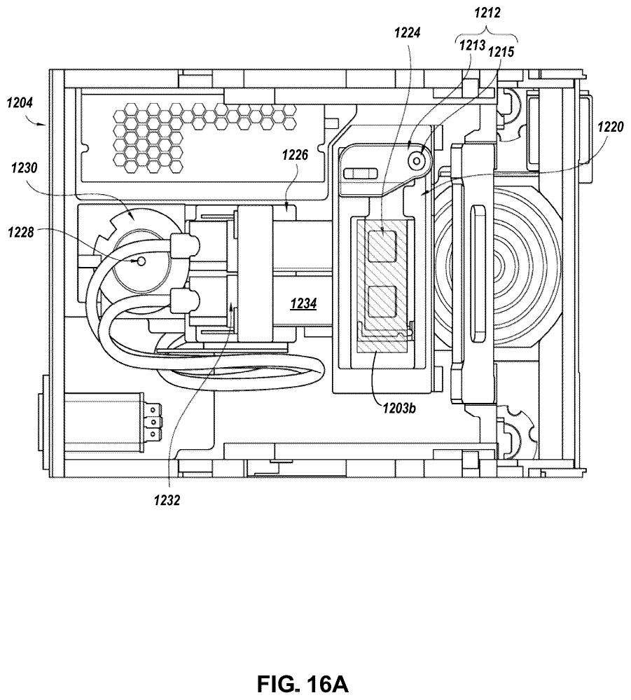

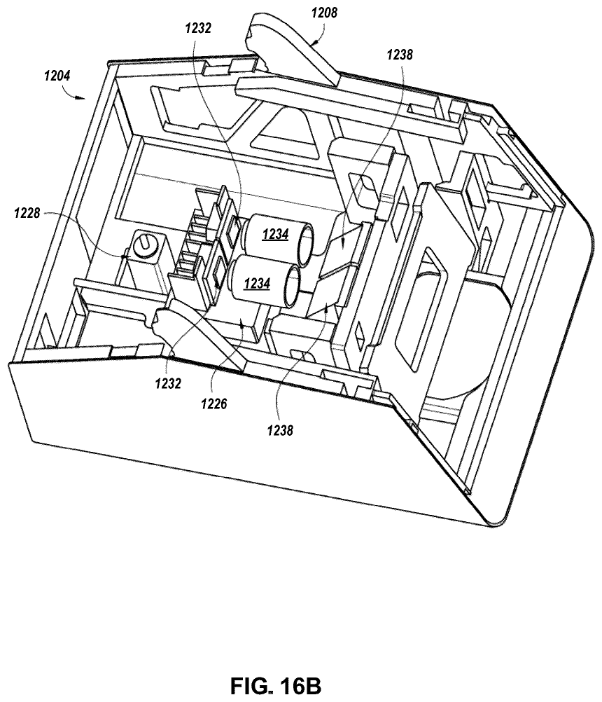

FIGS. 13-16B illustrate the lower housing 1204 of the SHS 1200 described in relation to FIG. 12. The housing cover 1236 of the lower housing 1204 has been removed in FIGS. 14, 16A, and 16B. As shown in FIGS. 13-14, the lower housing 1204 includes a base support member 1220 configured atop a shroud 1222 of the lower housing 1204. In various embodiments, the base support member 1220 operates in conjunction with the second retaining mechanism 1212 to retain a substrate 1216 upon the base support member 1220. In various embodiments, the base support member 1220 includes one or more base windows 1224. In various embodiments, the base windows 1224 are in alignment with corresponding sample windows provided on sample support members 1240 that are arranged in the upper housing 1202 (and the upper housing 1202 and lower housing 1204 are in the closed configuration). The lower housing 1204 can include optional thermal element 1203b that can share the features of the thermal element 1203.

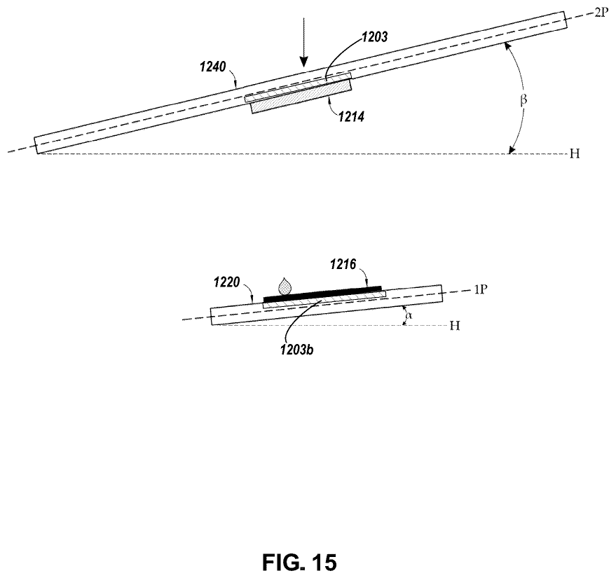

In various embodiments, the base support member 1220 defines a first plane 1P at a first angle α relative to a horizontal plane H when in the closed configuration (but not yet actuated into the sandwiched configuration), as shown in FIG. 15. In various embodiments, the first angle α is about 3 degrees, although other angles can be envisioned. In various embodiments, the first angle α is about 0.5 degree to about 30 degrees. For example, in some embodiments, the first angle α can be about 0.5, 1.0, 1.5, 2.0, 2.5, 3.0, 3.5, 4.0, 4.5, or 5.0 degrees relative to a horizontal plane.

For example, as shown in FIG. 15, the base support member 1220 defines a first plane 1P and includes a substrate 1216 having a barcoded capture array thereon. In various embodiments, the base support member 1220 is positioned at the first angle α relative to the horizontal plane H. In various embodiments, the sample support member 1240 defines a second plane 2P at an angle β relative to the horizontal plane H (thus having a second angle between second plane 2P and first plane 1P). In various embodiments, the second angle is determined as β minus α. In some embodiments, the angle β can be about 7 degrees, although other angles can be envisioned. In various embodiments, the angle β is about 1 degree to about 45 degrees. In some embodiments, the second angle can be about 0.5, 1.0, 1.5, 2.0, 2.5, 3.0, 3.5, 4.0, 4.5, 5.0, 5.5, 6.0, 6.5, 7, 7.5, 8, 8.5, 9, 9.5, or 10 degrees.

As shown in FIG. 16A, the shroud 1222 has been removed to show a top-view internal components of the lower housing 1204 (in a plan view). The lower housing 1204 includes a focus stage 1226 which can translate linearly toward or away from the base support member 1220 (when viewed in the plan view). In various embodiments, the focus stage 1226 is translated via actuation of a motor 1228 (e.g., a stepper motor) configured to cause a cam wheel 1230 to rotate so as to move the focus stage 1226 toward or away from the base support member 1220 relative to a horizontal plane on which the SHS 1200 is placed. In various embodiments, the cam wheel 1230 is coupled to a roller (not shown) that causes the focus stage to translate. In various embodiments, the roller is threaded. In various embodiments, the cam wheel 1230 has a first diameter and the roller has a second diameter thereby defining a gear ratio between the motor 1228 and the roller. As further shown in FIGS. 16A and 16B, the focus stage 1226 includes at least one image sensor 1232 and at least one lens or objective 1234 associated with each image sensor 1232 and positioned on the stage 1226. As shown in FIG. 16B, the base support member 1220 and the cam wheel 1230 have been removed to better illustrate the positioning of the motor 1228, the image sensors 1232, and the lenses 1234. In addition, the lower housing 1204 can also include one or more mirrors 1238 configured to reflect light, from an LED assembly configured in the upper housing 1202 which will be described later, toward the lenses 1234 and the image sensors 1232. In some embodiments, the mirrors 1238 are right-angle prisms, as shown in FIG. 16B.

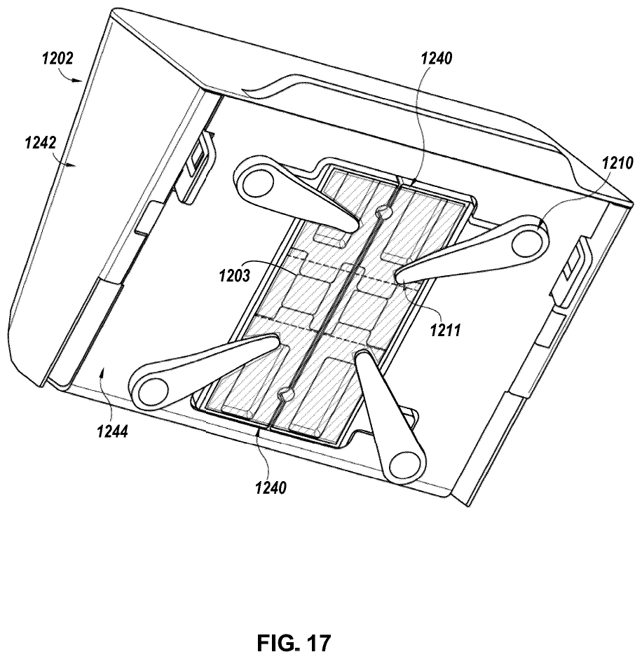

Referring to FIG. 17, the upper housing 1202 includes a housing cover 1242 and a shroud 1244 enclosing the upper housing 1202. As described above, the upper housing 1202 also includes one or more sample support members 1240 and the thermal element 1203. In various embodiments, each sample support member 1240 is configured to receive and retain a first substrate 1214 (e.g., a sample substrate) therein. In various embodiments, the retaining mechanisms 1210 are configured to apply a force onto the first substrate 1214 so as to maintain the first substrate 1214 within the sample support member 1240.

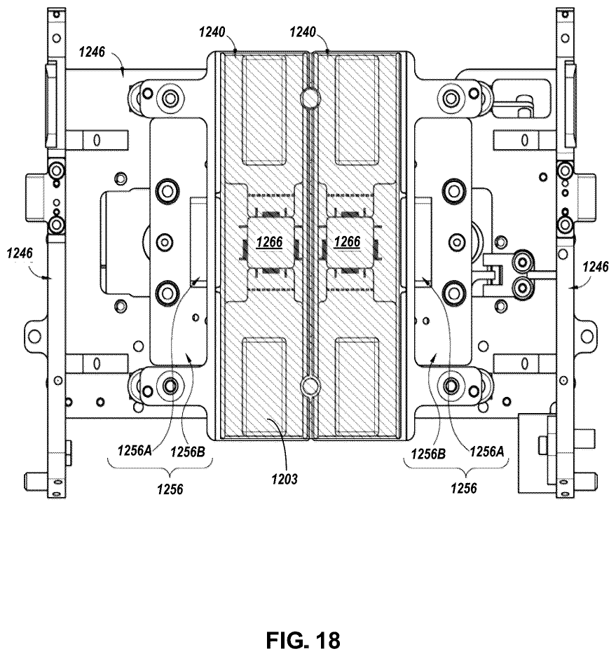

As shown in FIG. 18, each of the sample support members 1240 includes a sample window 1266. In various embodiments, the sample window 1266 is aligned with the base windows 1224 when the upper housing 1202 and the lower housing 1204 are in the closed configuration. For example, after the sandwiching operation is complete and the sample substrate contacts the spacer and/or the substrate having the barcoded capture array, each sample window 1266 aligns with a respective base window 1224 to allow for illumination and imaging of each sample. The thermal element 1203 can be transparent so that the thermal element 1203 does not obstruct each sample window 1266. In some embodiments, the thermal element 1203 can cover an area of each support member 1240 except for the sample windows 1266.

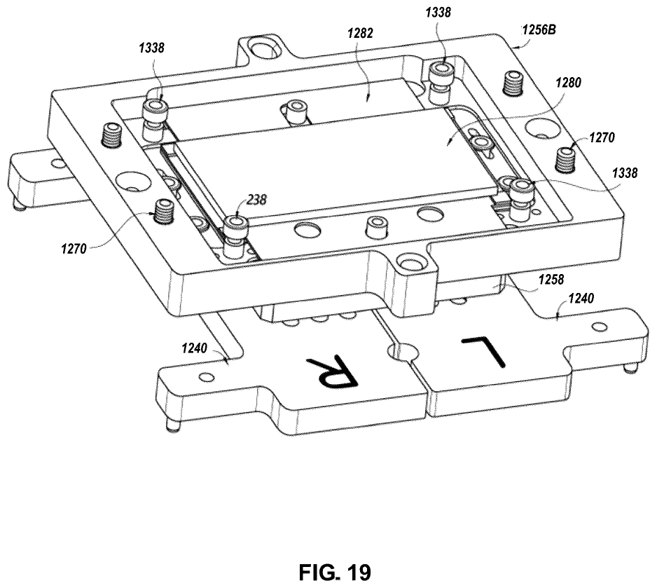

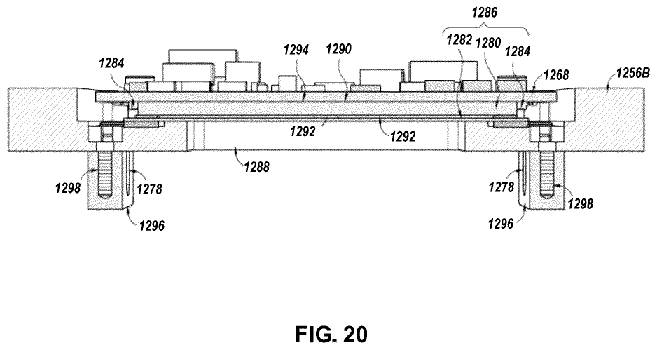

Referring to FIGS. 19 and 20, a printed circuit board (PCB) 168 is positioned between upper linear motion member 1256A and the lower linear motion member 1256B as shown in FIGS. 12 and 13. In various embodiments, the PCB 1268 includes one or more light emitting diodes. In various embodiments, the upper linear motion member 1256A is coupled to the lower linear motion member 1256B by one or more attachment means 1270. In various embodiments, the lower linear motion member 1256B includes receiving elements 1336 configured to receive bolts 1338 for securing the PCB 1268 to the lower linear motion potion 1256B.

As shown in FIGS. 19 and 20, the upper housing 1202 can also include a light emitting diode (LED) assembly 1286. In various embodiments, the LED assembly 1286 is configured to provide light through an aperture 1288 of the lower linear motion member 1256B as shown in FIG. 20. In various embodiments, the LED assembly 1286 is positioned within a recess of the lower linear motion member 1256B. In various embodiments, the LED assembly 1286 is positioned to substantially extend across the entire aperture 1288 (i.e., an area of illumination of the LED assembly substantially equals or is greater than an area of the aperture). In various embodiments, the LED assembly 1286 is positioned below the PCB 1268 as shown in FIG. 20. In various embodiments, the LED assembly 1286 includes a light guide 1280, a diffuser 1282 positioned below the light guide 1280, and one or more LEDs 1284 arranged around a periphery of the light guide 1280, such as adjacent to a side wall or a side surface of the light guide 1280. In various embodiments, an area of the light guide 1280 is substantially equal to or greater than an area of the aperture 1288, although in some embodiments, the area of the light guide 1280 can be greater than or less than the area of the aperture 1288. In some embodiments, the LED assembly 1286 is configured to emit light through the thermal element 1203 and/or the thermal element 1203b.

In various embodiments, the light guide 1280 includes a top surface 1290 that is opposite a bottom surface 1292. In various embodiments, the diffuser 1282 is positioned at a predetermined distance from the bottom surface 1292. In various embodiments, the diffuser 1282 is positioned in contact with the bottom surface 1292. In various embodiments, the diffuser 1282 can be spaced apart from the light guide 1280. The diffuser 1282 can include an aperture in which a cylindrical threaded bushing 1293 can be positioned. The bushing 1293 can extend from the lower linear motion member 1256B toward the PCB 1268. A screw or similar attachment means can be received through a hole in the PCB 1268 into the bushing 1293. In various embodiments, the top surface 1290 includes a reflective layer 1294 configured to reflect light emitted from the LEDs 1284 (and transmitted into the light guide 1280) through the bottom surface 1292, the diffuser 1282, and into the aperture 1288. In various embodiments, the reflective layer 1294 includes a metal. For example, the reflective layer can include a metal sheet or metal film. In some embodiments, the reflective layer 1294 can include a coating. For example, the reflective layer 1294 can include a silvered surface. As another example, the reflective layer 1294 can include a sputtered metal coating. In some embodiments, the coating can include nanoparticles that can be disposed in a matrix. For example, the nanoparticles can include metallic oxide nanoparticles, such as aluminum oxide nanoparticles or titanium dioxide nanoparticles. In some embodiments, the mean diameter of the nanoparticles is less than or equal to 600 nanometers, although other diameter sizes can be envisioned. In some embodiments, the matrix includes an epoxy polymer.

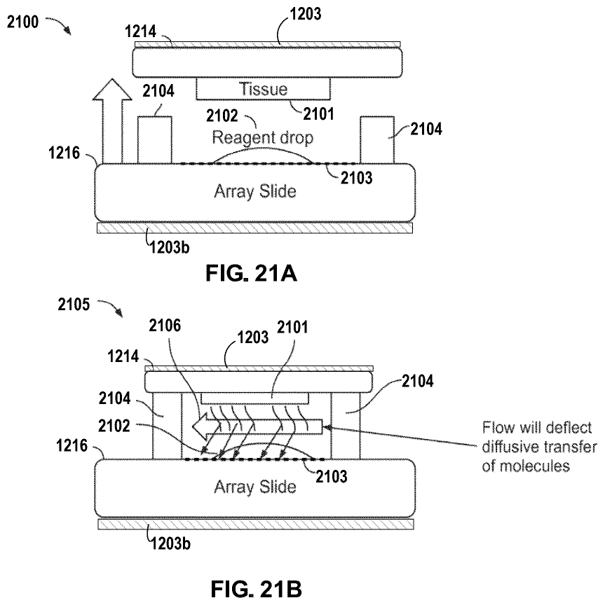

In various embodiments, the tilt member 1258 is rotatably coupled to the lower linear motion member 1256B via a plurality of arms 1296, such as arms 1296A and 1296B as shown in FIG. 20. In various embodiments, the arms 1296 are coupled to the lower linear motion member 1256B via bolts 1298 as shown in FIGS. 19 and 20. In various embodiments, the tilt member 1258 includes at least one pivot. For example, the tilt member 1258 includes two pivots 1278A, 1278B. FIGS. 21A and 21B show an exemplary sandwiching process 2100 where a first substrate (e.g., slide 1214), including a biological sample 2101, and a second substrate (e.g., array slide 1216 including an array 2103 having spatially barcoded capture probes) are brought into proximity with one another. As shown in FIG. 21A a liquid reagent drop (e.g., permeabilization solution 2102) is introduced on the second substrate in proximity to the capture probes of the array 2103 and in between the biological sample 2101 and the second substrate (e.g., slide 1216 including an array having spatially barcoded capture probes 2103). The permeabilization solution 2102 can release analytes or analyte derivatives (e.g., intermediate agents; e.g., ligation products) that can be captured by the capture probes of the array 2103. While a permeabilization solution 2102 is illustrated in FIGS. 21A and 21B, the permeabilization solution 2102 can be optional. For example, the thermal elements 1203 and/or 1203b can facilitate temperature control including a heating process or an off-mode that facilitates the thawing of the frozen tissue sample (e.g., tissue 2101). The thawing process can release analytes or analyte derivatives. As described in detail above, the thermal elements 1203 and 1203b can also control the temperature (e.g., in a cooling mode or heating mode) of the slide 1214 and slide 1216.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 1216) is in an inferior position to the first substrate (e.g., slide 1214). In some embodiments, the first substrate (e.g., slide 1214) can be positioned superior to the second substrate (e.g., slide 1216). A reagent medium 2102 within a gap between the first substrate (e.g., slide 1214) and the second substrate (e.g., slide 1216) creates a liquid interface between the two substrates. In some embodiments, the reagent medium can be a permeabilization solution which permeabilizes and/or digests the biological sample 2101. In some embodiments wherein the biological sample 2101 has been pre-permeabilized, the reagent medium is not a permeabilization solution. Herein, the reagent medium can also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 2101 can release from the biological sample, and actively or passively migrate (e.g., diffuse) across the gap toward the capture probes on the array 2103. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788, and US. Patent Application Pub. No. 2021/0189475, each of which is hereby incorporated by reference.

As further shown, one or more spacers 2104 can be positioned between the first substrate (e.g., slide 1214) and the second substrate (e.g., array slide 1216 including the array 2103 of spatially barcoded capture probes). The one or more spacers 2104 can be configured to maintain a separation distance between the first substrate and the second substrate. While the one or more spacers 2104 is shown as disposed on the second substrate, the spacer can additionally or alternatively be disposed on the first substrate.

In some embodiments, the one or more spacers 2104 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance can include a distance of at least 2 μm.

FIG. 21B shows a fully formed sandwich configuration 2105 creating a chamber 2106 formed from the one or more spacers 2104, the first substrate (e.g., the slide 1214), and the second substrate (e.g., the slide 1216 including an array 2103 having spatially barcoded capture probes) in accordance with some example implementations. In the example of FIG. 21B, a liquid reagent (e.g., the permeabilization solution 2102) fills the volume of the chamber 2106 and can create a permeabilization buffer that allows analytes (e.g., mRNA transcripts and/or other molecules) or analyte derivatives (e.g., intermediate agents; e.g., ligation products) to diffuse from the biological sample 2101 toward the capture probes of the second substrate (e.g., slide 1216). In some aspects, flow of the permeabilization buffer can deflect transcripts and/or molecules from the biological sample 2101 and can affect diffusive transfer of analytes or analyte derivatives (e.g., intermediate agents; e.g., ligation products) for spatial analysis. A partially or fully sealed chamber 2106 resulting from the one or more spacers 2104, the first substrate, and the second substrate can reduce or prevent flow from undesirable convective movement of transcripts and/or molecules over the diffusive transfer from the biological sample 2101 to the capture probes.

In some embodiments, a reagent medium (e.g., 1002 in FIG. 21A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of sodium dodecyl sulfate (SDS) or a sodium salt thereof, proteinase K, pepsin, N-lauroylsarcosine, and RNase.

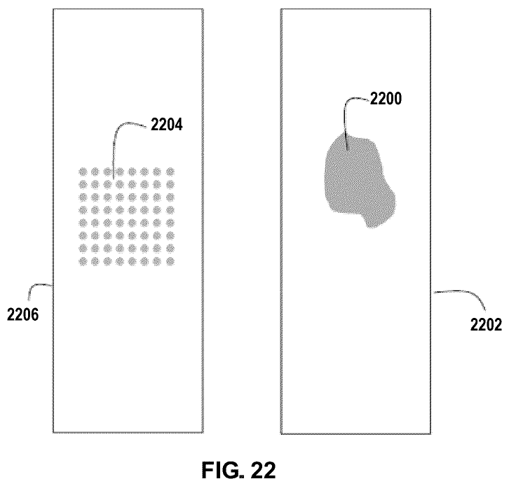

FIG. 22 is a schematic diagram showing a sample 2200 mounted on a first substrate 2202, and a feature array 2204 on a second substrate 2206. In some embodiments, the sample 2200 (and the first substate 2202) can be temperature controlled. For example, the thermal element 1203 and/or thermal element 1203b can be implemented to control the temperature of the first substrate 2202 and/or the second substrate 2206.

In some workflows, the alignment and contacting operations are performed manually. However, manual alignment is prone to operator error and inconsistency: alignment operations between sample 2200 and feature array 2204 may be inconsistent and/or imperfect. Improper alignment of sample 2200 and feature array 2204 can be disadvantageous for a number of reasons. For example, if the sample and array are imperfectly aligned when contact occurs, it may not be possible to successfully remove the sample and attempt re-alignment, and the array may be rendered unusable. For expensive feature arrays, this results in significant increased assay cost.

Further, certain assays involving imaging the sample through the feature array. If the sample and array are improperly aligned, imaging can be imperfect, and can be adversely affected by imperfections that arise from mis-alignment.

In addition, many tissue samples are available in archived (i.e., slide mounted) form. As a result, workflows that rely on direct physical placement of the tissue sample on a feature array may not be able to accommodate such samples. This limits the applicability of such workflows to only a subset of available samples.

The present disclosure features devices and methods for alignment of a sample 2200 and a feature array 2204. The devices and methods ensure correct alignment and contact between the sample and feature array so that reproducible spatial analyses can be conducted in a manner that is not significantly affected by systematic variations in alignment errors. The devices and methods reduce consumables waste (i.e., wasted feature arrays) and cost, and also reduce sample waste. In some embodiments, feature array 2204 is includes printed spots, barcoded gels, barcoded microspheres, a gel film, or any combination thereof. The feature array 2204 can also be a uniform coating of probes, such as in tissue optimization slides.

As described above, a feature array 2204 can be positioned on second substrate 2206. More generally, however, second substrate 2206 supports a reagent medium that is used to analyze sample 2200. In some embodiments, the reagent medium corresponds to feature array 2204. In certain embodiments, the reagent medium includes feature array 2204 and one or more additional components. For example, the additional components can include a permeabilization reagent (e.g., a solid, liquid, gel, or dried permeabilization reagent). As an additional example, the additional components can include a hydrogel compound or layer with an embedded permeabilization reagent.

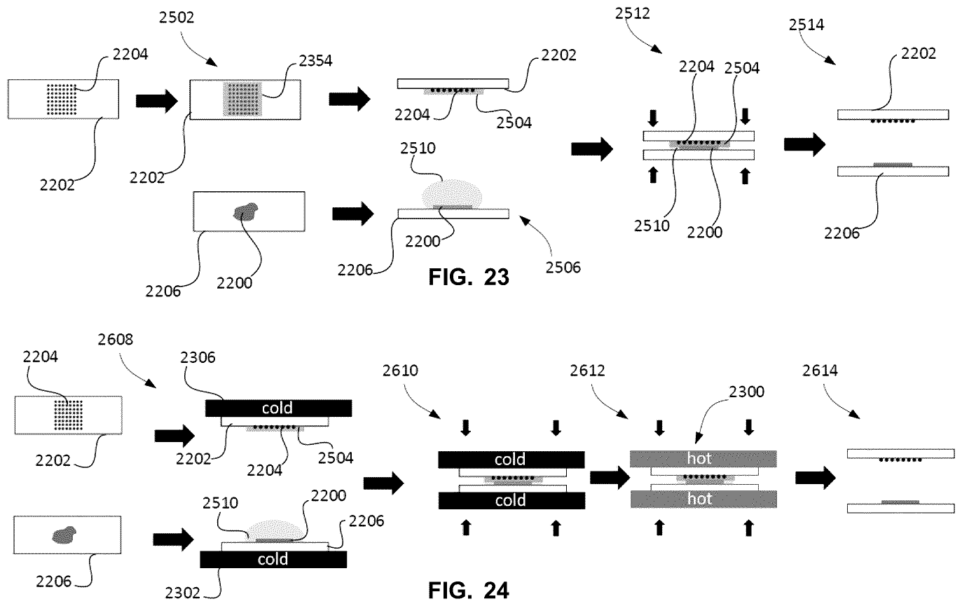

FIG. 23 shows an example analytical workflow using dried permeabilization reagents 1401 on first substrate 2202, which includes a feature array 2204. In a first step 2502, the first substrate 2202 can be coated with a dried permeabilization reagent 2204. As mentioned above, the permeabilization reagent can be deposited in solution on first substrate 2202 and then dried. Alternatively, in other embodiments, the permeabilization reagent can be applied in dried form directly onto the first substrate 2202. In some embodiments, the dried permeabilization reagent 2204 covers at least the surface area of the first substrate 2202 that includes the feature array 2204. In some embodiments, the dried permeabilization reagent 2204 covers a surface area of the first substrate 2202 that is greater than the surface area which includes the feature array 2204. In some embodiments, the dried permeabilization reagent 2204 is dried pepsin. In some embodiments, the dried permeabilization reagent 2204 is a dried permeabilization enzyme, a dried buffer, a dried detergent, or any combination thereof. In some embodiments, the dried detergent is dried octyl phenol ethoxylate.

In a second step 2506, the second substrate 2206, which includes a sample 2200 (e.g., a histological tissue section) is hydrated. The sample 2200 can be hydrated by contacting the sample 2200 with a buffer 1210 that does not include a permeabilization reagent. In some embodiments, buffer 2510 is hydrochloric acid. In some embodiments, buffer 2510 is a solvent. In some embodiments, buffer 2510 is a permeabilization buffer that does not contain any permeabilization reagents. For example, the sample 2200 on second substrate 2206 can be hydrated in hydrochloric acid, and first substrate 2202 can be coated with dried pepsin.

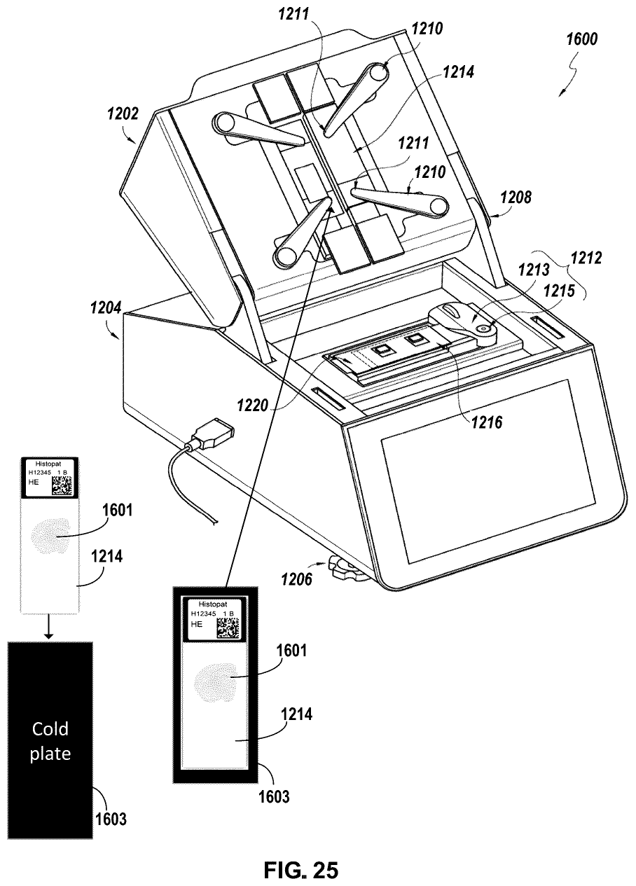

In a third step 2512, the first substrate 2202 and the second substrate 2204 can be arranged in a sandwich assembly, as shown in FIG. 25. The dried permeabilization reagent 2504 on the first substrate 2202 is solubilized when in contact with the buffer 2510. In some embodiments, the tissue permeabilization process begins when the sample 2200 and the dried permeabilization reagent 2504 are contacted with the buffer 2510. During the permeabilization process, analytes can be released from the sample 2200. In some embodiments, analytes that are released from the permeabilized sample diffuse to the surface of the first substrate 2202 and are captured on the feature array 2204 (e.g., on barcoded probes). In some embodiments, first substrate 2202 can be placed in direct contact with the sample 2200 on the second substrate 2204, ensuring no diffusive spatial resolution losses.

In a fourth step 2514, the first substrate 2202 and the second substrate 2206 are separated (e.g., pulled apart). In some embodiments, the sample analysis (e.g., cDNA synthesis) can be performed on the first substrate 2202 after the first substrate 2202 and the second substrate 2206 are separated. In some embodiments, the second substrate 2206 can be discarded or archived after the first substrate 2202 and the second substrate 2206 are separated.

An analytical workflow may be substantially similar in several aspects to the example analytical workflow shown in FIG. 23 discussed above, but can include an alternative methods to control a temperature of the first and second substrates 2202 and 2206 throughout the analytical process instead of not controlling temperature. FIG. 24 shows an example analytical workflow using temperature-controlled first and second members 2302 and 2306, respectively, of sample holder 2300. In some embodiments, the temperature of the first and second members 2302 and 2306 is lowered to a first temperature that is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower). In some embodiments, the sample holder 2300 includes a temperature control system (e.g., heating and cooling conducting coils) that enables a user to control the temperature of the sample holder 2300. Alternatively, in other embodiments, the temperature of the sample holder 2300 is controlled externally (e.g., via refrigeration or a hotplate). In a first step 2608, the second member 2306, set to or at the first temperature, contacts the first substrate 2202, and the first member 2302, set to or at the first temperature, contacts the second substrate 2204, thereby lowering the temperature of the first substrate 2202 and the second substrate 2204 to a second temperature. In some embodiments, the second temperature is equivalent to the first temperature. In some embodiments, the first temperature is lower than room temperature (e.g., 25 degrees Celsius). In some embodiments, the second temperature ranges from about −10 degrees Celsius to about 4 degrees Celsius. In some embodiments, the second temperature is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower).

The first substrate 2202, which includes a feature array 2204, is contacted with the dried permeabilization reagent 2204. In some embodiments, the first substrate 2202 is contacted with a permeabilization reagent that is a gel or a liquid. Also in first step 2608, sample 2200 is contacted with buffer 2510. Both first and second substrates 2202 and 2206 are placed at lower temperature to slow down diffusion and permeabilization efficiency. Alternatively, in some embodiments, the sample 2200 can be contacted directly with a liquid permeabilization reagent without inducing an unwanted initiation of permeabilization due to the substrates being at the second temperature. In some embodiments, the low temperature slows down or prevents the initiation of permeabilization.

In a second step 2610, keeping the sample holder 2300 and substrates at a cold temperature (e.g., at the first or second temperatures) continues to slow down or prevent the permeabilization of sample 2200. In a third step 2612, the sample holder 2300 (and consequently the first and second substrates 2202 and 2206) is heated up to initiate permeabilization. In some embodiments, the sample holder 2300 is heated up to a third temperature. In some embodiments, the third temperature is above room temperature (e.g., 25 degrees Celsius) (e.g., 30 degrees Celsius or higher, 35 degrees Celsius or higher, 40 degrees Celsius or higher, 50 degrees Celsius or higher, 60 degrees Celsius or higher). In some embodiments, analytes that are released from the permeabilized tissue of sample 2200 diffuse to the surface of the first substrate 2202 and are captured on the feature array 2204 (e.g., barcoded probes) of the second substrate 2206. In a fourth step 2614, the first substrate 2202 and the second substrate 2206 are separated (e.g., pulled apart) and temperature control is stopped.

In some embodiments, where either first substrate 2202 or substrate second 2206 (or both) includes wells, a permeabilization solution can be introduced into some or all of the wells, and then sample 2200 and feature array 2204 can be contacted by closing sample holder 2300 to permeabilize sample 2200. In certain embodiments, a permeabilization solution can be soaked into a hydrogel film that is applied directly to sample 2200, and/or soaked into features (e.g., beads) that form feature array 2204. When sample 2200 and feature array 2204 are contacted by closing sample holder 2300, the permeabilization solution promotes migration of analytes from sample 2200 to feature array 2204.

In certain embodiments, different permeabilization agents or different concentrations of permeabilization agents can be infused into array features (e.g., beads) or into a hydrogel layer as described above. By locally varying the nature of the permeabilization reagent(s), the process of analyte capture from sample 2200 can be spatially adjusted.

It should also be noted that in connection with any of the above permeabilization methods, in some embodiments, migration of the permeabilization agent into sample 2200 can be passive (e.g., via diffusion). Alternatively, in certain embodiments, migration of the permeabilization agent into sample 2200 can be performed actively (e.g., electrophoretic, by applying an electric field to promote migration).

In certain embodiments, different permeabilization agents or different concentrations of permeabilization agents can be infused into array features (e.g., beads) or into a hydrogel layer as described above. By locally varying the nature of the permeabilization reagent(s), the process of analyte capture from sample 2200 can be spatially adjusted.

It should also be noted that in connection with any of the above permeabilization methods, in some embodiments, migration of the permeabilization agent into sample 2200 can be passive (e.g., via diffusion). Alternatively, in certain embodiments, migration of the permeabilization agent into sample 2200 can be performed actively (e.g., electrophoretic, by applying an electric field to promote migration).

Referring to FIG. 25, another example embodiment of a sample handling system (SHS) 1600 is shown. The SHS 1600 can share features with the SHS 1200 and each SHS 1200, 1600 can operate in a similar or same manner. In some embodiments, the SHS 1600 does not include the thermal element 1203 in the upper housing 1202. The SHS 1600 can be configured to receive one or more first substrates 1214 (e.g., two first substrates) in the upper housing 1202 which can secure the first substrates 1214 with the first retaining mechanisms 1210 as described above. A sample 1601 (e.g., a tissue sample, a biological sample) can be mounted on the first substrate 1214 as described above. As illustrated in FIG. 25, prior to insertion into the SHS 1600, the first substrate 1214 can be positioned on a thermal element 1603 that is separate from the SHS 1600. The thermal element 1603 can operate in a similar or same manner to thermal element 1203 above. In some embodiments, the thermal element 1603 is a cold plate, a cooling plate, or otherwise temperature controlled plate, slide, plane, or other surfaces that are temperature controlled (e.g., to cool and optionally to heat). The temperature control of the thermal element 1603 can facilitate transfer of temperature control from the thermal element 1603 to other surfaces that contact the thermal element (e.g., the first substrate 1214). The thermal element 1603 can control a temperature of the first substrate 1214. In some embodiments, the heating effect can facilitate the thawing or release of a frozen tissue sample and targets within the sample. For example, the thermal element 1603 is configured to maintain a surface temperature of the first substrate 1214 between −80° C. and 5° C. (e.g., −20° C. and 5° C.) (e.g., a cooling and/or freezing effect). The thermal element 1603 is configured to maintain a surface temperature of the first substrate 1214 around 0° C. (e.g., a cooling and/or freezing effect).

Some embodiments include inserting the temperature controlled first substrate 1214 (e.g., flash frozen and kept frozen by thermal element 1603) alone into the SHS 1200. In such examples, the first substrate 1214 is kept at a threshold temperature before sandwiching and capturing is completed at the SHS 1600. In some embodiments, the cooling effect can facilitate the freezing of or keeping frozen of a tissue sample and targets within the sample. In some embodiments, the heating effect can facilitate the thawing or release of a frozen tissue sample and target analytes, or proxies thereof, within the biological sample. For example, the thermal element 1603 is configured to maintain a surface temperature of the first substrate 1214 between −20° C. and 5° C. (e.g., a cooling and/or freezing effect). The thermal element 1603 is configured to maintain a surface temperature of the first substrate 1214 around 0° C. (e.g., a cooling and/or freezing effect).

Some embodiments include inserting the assembly of the temperature controlled first substrate 1214 and the thermal element 1603 together into the SHS 1600. The assembly of the thermal element 1603 and the first substrate 1214 can be inserted into the upper housing 1202 of the SHS 1600 in a same or similar manner as described above for the insertion of the first substrate 1214 into the SHS 1200. The thermal element 1603 can continue to control the temperature of the first substrate 1214 after insertion of the assembly of the first substrate 1214 and the thermal element 1603 into the thermal element 1603 can share features with the thermal element 1203, and can be a thin, transparent plate that is configured to control the temperature of the first substrate 1214 and facilitate imaging through the thermal element 1603 within the SHS 1600. Some embodiments of the thermal element 1603 can facilitate cooling and heating in a similar manner as described above in relation to the thermal element 1203.

Any one of the systems and/or devices provided herein can include one or more housings, e.g., upper and lower housings, configured to receive a sample substrate. The sample substrate can include a sample, such as a biological sample. The biological sample can be a tissue sample, such as tissue section, e.g., a fresh frozen tissue section.

Any one of the systems and/or devices provided herein can include an alignment mechanism coupling the upper housing to the lower housing, or vice versa. In various embodiments, the alignment mechanism can be configured to move the upper housing, the lower housing, or both into an arrangement having an open configuration, a closed configuration, or an intermediate configuration therebetween.

The upper housing and/or the lower housing can include a thermal element configured to control a temperature of the sample substrate. In some embodiments, the thermal element is mounted to the upper housing between the upper housing and the sample substrate. In some embodiments, the thermal element is mounted to the upper housing and surrounds the sample substrate. In some embodiments, the lower housing can include a lower housing thermal element that controls a temperature at a base support member of the lower housing.

The thermal element can be configured to cool or heat the sample substrate. In some embodiments, the thermal element includes liquid nitrogen. In some embodiments, the thermal element can include a cooling liquid that circulates within the system to cool the sample substrate. The thermal element can optionally be transparent. Examples of a thermal element include, but are not limited to, a thermoelectric cooler (TEC) and a cooling plate.

The thermal element can be configured to activate or deactivate based on the configuration of the system. For example, in the closed configuration, the thermal element can be configured to deactivate. The thermal element can be configured to, upon energizing, maintain a surface temperature of the sample substrate between −20° C. and 5° C. in the system. In some embodiments, the thermal element is configured to maintain a surface temperature of the sample substrate around 0° C. In some cases, the thermal element is configured to, upon energizing the thermal element, maintain a surface temperature of the sample substrate between 10° C. and 40° C. In some cases, the thermal element is configured to, upon energizing the thermal element in a cooling mode, maintain a surface temperature of the sample substrate between −80° C. and 5° C. (e.g., −20° C. and 5° C.). In some embodiments, the thermal element is configured to, upon energizing the thermal element in a heating mode, maintain a surface temperature of the sample substrate between 10° C. and 40° C.

In some embodiments, the sample substrate can include a sample window. The upper housing can optionally include a light emitting diode (LED) assembly, wherein the LED assembly is configured to emit light through the sample window of the sample substrate. The LED assembly can be configured to emit light through the thermal element. The sample substrate can also include at least one base window. One or more of the base windows can configured to align with the sample window in the closed configuration.

Any of one of the methods described herein can include connecting a substrate to an upper housing. The substrate can include a biological sample. Any one of the methods provided herein can include cooling, by a thermal element of the upper housing, the substrate such that the biological sample is at or below a threshold temperature. In various embodiments, the thermal element can be configured to cool the substrate.

Any of the methods provided herein can include aligning the upper housing with a lower housing by an alignment mechanism. The alignment mechanism can be configured to move the upper housing, the lower housing, or both into an arrangement having an open configuration, a closed configuration, or an intermediate configuration therebetween. In some embodiments, the methods described herein can include moving the upper housing towards the lower housing.

Any of the methods provided herein can include deactivating the thermal element at the upper housing; releasing one or more analytes from the biological sample; and capturing the one or more analytes from the biological sample onto a spatial array disposed on the lower housing. The methods provided herein can deactivate the thermal element responsive to moving the upper housing from the open configuration into the closed configuration. In some embodiments, the methods provided herein can align a base window at a base support member with a sample window at the sample support member. The methods herein can include maintaining a surface temperature of the sample substrate between −20° C. and 5° C. The threshold temperature can include a range of about −5° C. and 5° C., inclusive.

Any of the methods provided herein can include a method for analyzing an analyte in a biological sample mounted on a first substrate. The method can include:
(a) controlling a temperature of the first substrate in a cooling mode to maintain a fixed state of the biological sample on the first substrate;
(b) contacting the biological sample with at least one analyte capture agents, wherein an analyte capture agent comprises a capture agent barcode domain, wherein the capture agent barcode domain comprises an analyte binding moiety barcode and a capture handle sequence;
(c) aligning the first substrate with a second substrate comprising an array, such that at least a portion of the biological sample is aligned with at least a portion of the array, wherein the array comprises a plurality of capture probes, wherein a capture probe of the plurality of capture probes comprises: (i) a spatial barcode and (ii) a capture domain;
(d) when the biological sample is aligned with at least a portion of the array, (i) releasing the capture agent barcode domain from the analyte by controlling the temperature of the first substrate in either a heating mode or an off mode; and (ii) passively or actively migrating the capture agent barcode domain from the biological sample to the array; and
(e) coupling the capture handle sequence to the capture domain.

Any of the methods provided herein can include determining (i) all or a part of the sequence of the capture agent barcode domain, or a complement thereof; and (ii) the sequence of the spatial barcode, or a complement thereof. The methods herein can optionally include using the determined sequence of (i) and (ii) to determine a location of the analyte in the biological sample. The determining step can include sequencing (i) all or a part of the capture agent barcode domain, or a complement thereof, and (ii) the sequence of the spatial barcode, or a complement thereof.

Any of the methods provided herein can include a method for analyzing a nucleic acid analyte in a biological sample mounted on a first substrate. The method can include:
(a) controlling a temperature of the first substrate in a cooling mode to maintain a fixed state of the biological sample on the first substrate;
(b) contacting the biological sample with a first probe and a second probe, wherein the first probe and second probe hybridize to the nucleic acid analyte, wherein the first probe and the second probe each comprise a sequence that is substantially complementary to a first and second sequence of the nucleic acid analyte, and wherein the second probe oligonucleotide comprises a capture probe binding domain;
(c) coupling the first probe and the second probe, thereby generating a connected probe;
(d) aligning the first substrate with a second substrate comprising an array, such that at least a portion of the biological sample is aligned with at least a portion of the array, wherein the array comprises a plurality of capture probes, wherein a capture probe of the plurality of capture probes comprises: (i) a spatial barcode and (ii) a capture domain;
(e) when the biological sample is aligned with at least a portion of the array, (i) releasing the connected probe from the nucleic acid analyte by controlling the temperature of the first substrate in either a heating mode or an off mode; and (ii) passively or actively migrating the connected probe from the biological sample to the array; and
(f) hybridizing the connected probe to the capture domain via the capture probe binding domain.

In some embodiments, the first and second sequence of the nucleic acid analyte abut one another or are at least 1, 2, 3, 4, 5, 6, 7, 8, 9, 10, or more nucleotides away from one another.

In some embodiments, the releasing step, the aligning step, and/or step (e) can include contacting the biological sample and the array with a reagent medium comprising a nuclease, a permeabilization agent, or a combination thereof. In some embodiments, the analyte comprises a protein or a fragment thereof, or a peptide. The releasing step can include simultaneously permeabilizing the biological sample and releasing the capture agent barcode domain from the analyte binding moiety, or simultaneously permeabilizing the biological sample and releasing the connected probe from the nucleic acid analyte.

In some embodiments, the reagent medium includes a detergent. The detergent can be selected from sodium dodecyl sulfate (SDS), sarkosyl, saponin, polyethylene glycol tert-octylphenyl ether, or polysorbate 20. Further, the reagent medium can include less than 15 w/v % of a detergent selected from SDS and sarkosyl, or optionally the reagent medium includes as least 5% w/v % of a detergent selected from SDS and sarkosyl. In some embodiments, the reagent medium does not include a detergent and/or a permeabilization agent, preferably the reagent medium does not include sodium dodecyl sulfate (SDS) or sarkosyl. The reagent medium can further include one or more crowding agents, optionally PEG.

In some embodiments, the coupling the first probe and the second probe comprises ligating the first probe and the second. In some embodiments, the ligating can include the use of a ligase, optionally wherein the ligase is selected from a T4 RNA ligase (Rnl2), a PBCV-1 DNA Ligase or Chlorella virus DNA Ligase, a single-stranded DNA ligase, and a T4 DNA ligase. The capture probe can include a poly(T) sequence. The capture probe can include a sequence complementary to the capture handle sequence or the capture probe binding domain. Further, the capture probe can include one or more functional domains, a unique molecular identifier (UMI), a cleavage domain, or a combination thereof. The analyte binding moiety can include an antibody. The analyte capture agent can include a linker, optionally wherein the linker is a cleavable linker. The cleavable linker can be a photo-cleavable linker, a UV-cleavable linker, or an enzyme cleavable linker. The nucleic acid analyte can be RNA or DNA, preferably the RNA is mRNA.

Any of the methods provided herein can include an aligning step that includes: (i) mounting the first substrate on a first member of a support device, the first member configured to retain the first substrate; (ii) mounting the second substrate on a second member of the support device, (iii) applying the reagent medium to the first substrate and/or the second substrate, and (iv) operating an alignment mechanism of the support device to move the first member and/or the second member such that at least a portion of the biological sample is aligned with at least a portion of the array, and such that the portion of the biological sample and the portion of the array contact the reagent medium. In some embodiments, the alignment mechanism is coupled to the first member, the second member, or both the first member and the second member. In some embodiments, the alignment mechanism includes a linear actuator, optionally wherein: the linear actuator is configured to move the second member along an axis orthogonal to a plane or of the first member and/or the second member, and/or the linear actuator is configured to move the first member along an axis orthogonal to the plane of the first member and/or the second member, and/or the linear actuator is configured to move the first member, the second member, or both the first member and the second member at a velocity of at least 0.1 mm/sec, and/or the linear actuator is configured to move the first member, the second member, or both the first member and the second member with an amount of force of at least 0.1 lbs.

Any of the methods provided herein can include, in the releasing step, a separation distance that is maintained between the first substrate and the second substrate, optionally wherein the separation distance is less than 50 microns, optionally wherein the separation distance is between 2-25 microns, optionally wherein the separation distance is measured in a direction orthogonal to a surface of the first substrate that supports the biological sample, and/or at least the portion of the biological sample is vertically aligned with at least a portion of the array. In some embodiments, at least one of the first substrate and the second substrate further includes a spacer, wherein after the first and second substrate being mounted on the support device, the spacer is disposed between the first substrate and second substrate and is configured to maintain the reagent medium within a chamber formed by the first substrate, the second substrate, and the spacer, and maintain a separation distance between the first substrate and the second substrate, the spacer positioned to at least partially surround an area on the first substrate on which the biological sample is disposed and/or the array disposed on the second substrate, wherein the area of the first substrate, the spacer, and the second substrate at least partially encloses a volume comprising the biological sample.

Any of the systems described herein (or a kit) for analyzing an analyte in a biological sample can include:
(a) a support device configured to retain a first substrate and a second substrate, wherein abiological sample is placed on the first substrate, and wherein the second substrate comprises an array including a plurality of capture probes, wherein a capture probe of the plurality of capture probes comprises: (i) a spatial barcode and (ii) a capture domain;
(b)
   (b1) a first probe and a second probe, wherein the first probe and the second probe each include a sequence that is substantially complementary to adjacent sequences of the analyte, wherein the second probe includes a capture probe binding domain, and wherein the first probe and the second probe are capable of being ligated together to form a connected probe; or
   (b2) a plurality of analyte capture agents, wherein an analyte capture agent of the plurality of analyte capture agents includes an analyte binding moiety and a capture agent barcode domain, wherein the analyte binding moiety specifically binds to the analyte in the biological sample, and wherein the capture agent barcode domain includes an analyte binding moiety barcode and an capture handle sequence;
(c) a reagent medium that includes an agent for releasing the connected probe and optionally a permeabilization reagent; and
(d) instructions for performing any one of the methods described herein.

In any of the methods, systems, or kits provided herein, the permeabilization agent can include a protease, optionally the protease is selected from trypsin, pepsin, elastase, or Proteinase K. The agent for releasing the connected probe can include an RNAse, optionally wherein the RNAse is selected from RNase A, RNase C, RNase H, or RNase I. The systems or kits disclosed herein can include an alignment mechanism on the support device to align the first substrate and the second substrate. The alignment mechanism can include a linear actuator, wherein the first substrate includes a first member and the second substrate comprises a second member. In some embodiments, the linear actuator is configured to move the second member along an axis orthogonal to a plane or of the first member and/or the second member, and/or the linear actuator is configured to move the first member along an axis orthogonal to the plane of the first member and/or the second member, and/or the linear actuator is configured to move the first member, the second member, or both the first member and the second member at a velocity of at least 0.1 mm/sec, and/or the linear actuator is configured to move the first member, the second member, or both the first member and the second member with an amount of force of at least 0.1 lbs.

Any one of the systems or kits provided herein can include an upper housing configured to receive a sample substrate and a thermal element; a lower housing; and an alignment mechanism coupling the upper housing to the lower housing. The alignment mechanism can be configured to move the upper housing, the lower housing, or both into an arrangement having an open configuration, a closed configuration, or an intermediate configuration therebetween. The thermal element can be configured to cool the sample substrate below room temperature. In some embodiments, the thermal element is configured to cool the sample substrate after insertion of the sample substrate into the upper housing. The thermal element can include a cooling plate in some embodiments. The sample substrate can include a sample window.

The upper housing can include a light emitting diode (LED) assembly, wherein the LED assembly is configured to emit light through the sample window of the sample substrate. The LED assembly can be configured to emit light through the thermal element.

Any one of the methods provided herein can include: cooling, by a thermal element, a substrate comprising a biological sample such that the biological sample is at or below a threshold temperature; connecting a substrate to an upper housing; aligning the upper housing with a lower housing by an alignment mechanism, the alignment mechanism being configured to move the upper housing, the lower housing, or both into an arrangement having an open configuration, a closed configuration, or an intermediate configuration therebetween; and moving the upper housing towards the lower housing; wherein the thermal element is configured to cool the substrate.

In some embodiments, the methods provide herein include connecting the thermal element to the upper housing. The thermal element can include a cooling plate.

Any one of the systems or kits provided herein, the system or kit can include:
a thermal element;
an upper housing configured to receive a sample substrate;
a lower housing; and
an alignment mechanism coupling the upper housing to the lower housing, the alignment mechanism being configured to move the upper housing, the lower housing, or both into an arrangement having an open configuration, a closed configuration, or an intermediate configuration therebetween;
wherein, the thermal element is configured to cool the sample substrate before insertion of the sample substrate into the upper housing.

The description will be further understood by reference to the following numbered paragraphs:
1. A system or device comprising:
   an upper housing configured to receive a sample substrate, the upper housing comprising a thermal element configured to control a temperature of the sample substrate;
   a lower housing; and
   an alignment mechanism coupling the upper housing to the lower housing, the alignment mechanism being configured to move the upper housing, the lower housing, or both into an arrangement having an open configuration, a closed configuration, or an intermediate configuration therebetween;
   wherein the thermal element is configured to cool the sample substrate.
2. The system or device of claim 1, further comprising the sample substrate, wherein the thermal element is mounted between the upper housing and the sample substrate.
3. The system or device of claim 2, wherein the sample substrate comprises a sample window.
4. The system or device of claim 3, wherein the upper housing further comprises a light emitting diode (LED) assembly, wherein the LED assembly is configured to emit light through the sample window of the sample substrate.
5. The system or device of claim 4, wherein the LED assembly is configured to emit light through the thermal element.
6. The system or device of any one of claims 1-5, wherein the lower housing further comprises a focus stage, wherein the focus stage comprises a stage, a motor configured to translate the stage, and at least one image sensor positioned on the stage, and at least one lens positioned on the stage.
7. The system or device of claim 4, wherein the sample substrate comprises at least one base window, wherein each of the at least one base window is configured to align with the sample window in the closed configuration.
8. The system or device of any one of claims 1-7, wherein the thermal element is transparent.
9. The system or device of any one of claims 1-8, wherein the thermal element is mounted to the upper housing and surrounds the sample substrate.
10. The system or device of any one of claims 1-9, wherein the thermal element is a thermoelectric cooler (TEC).
11. The system or device of any one of claims 1-10, wherein, in the closed configuration, the thermal element is configured to deactivate.
12. The system or device of any one of claims 1-11, wherein the lower housing comprises a second thermal element that controls a temperature at a base support member of the lower housing.
13. The system or device of any one of claims 1-12, wherein the thermal element comprises liquid nitrogen.
14. The system or device of any one of claims 1-13, wherein the thermal element comprises a cooling liquid that circulates within the thermal element to cool the sample substrate.
15. The system or device of any one of claims 1-14, wherein the thermal element is configured to, upon energizing the thermal element, maintain a surface temperature of the sample substrate between −80° C. and 5° C.
16. The system or device of any one of claims 1-15, wherein the thermal element is configured to, upon energizing the thermal element, maintain a surface temperature of the sample substrate around 0° C.
17. The system or device of any one of claims 1-16, wherein the thermal element is configured to, upon energizing the thermal element, maintain a surface temperature of the sample substrate between 10° C. and 40° C.
18. The system or device of any one of claims 1-17, wherein the thermal element is configured to, upon energizing the thermal element in a cooling mode, maintain a surface temperature of the sample substrate between −80° C. and 5° C.; and wherein the thermal element is configured to, upon energizing the thermal element in a heating mode, maintain a surface temperature of the sample substrate between 10° C. and 40° C.
19. The system or device of any one of claims of 1-18, wherein the sample substrate comprises a biological sample.
20. The system or device of claim 19, wherein the biological sample comprises tissue sample, preferably wherein the tissue sample is a tissue section, optionally a fresh frozen tissue section or a fixed tissue section.
21. A method comprising: connecting a substrate to an upper housing, the substrate comprising a biological sample;
   cooling, by a thermal element of the upper housing, the substrate such that the biological sample is at or below a threshold temperature;
   aligning the upper housing with a lower housing by an alignment mechanism, the alignment mechanism being configured to move the upper housing, the lower housing, or both into an arrangement having an open configuration, a closed configuration, or an intermediate configuration therebetween; and
   moving the upper housing towards the lower housing.
22. The method of claim 21, comprising:
   deactivating the thermal element of the upper housing;
   releasing one or more analytes, or proxies thereof, from the biological sample; and
   capturing the one or more analytes, or proxies thereof, from the biological sample onto a spatial array disposed on the lower housing.
23. The method of claim 22, wherein step (f) comprises deactivating the thermal element responsive to moving the upper housing and the lower housing from the open configuration into the closed configuration.
24. The method of claim 22, wherein step (d) comprises aligning a base window at a base support member with a sample window at the sample support member.
25. The method of any one of claims 21-24, wherein step (b) comprises maintaining a surface temperature of the sample substrate between −80° C. and 5° C.
26. The method of any one of claims 21-25, wherein the threshold temperature is in a range of about −5° C. and 5° C., inclusive.
27. A method for analyzing an analyte in a biological sample mounted on a first substrate, the method comprising:
   (a) controlling a temperature of the first substrate in a cooling mode to maintain a fixed state of the biological sample on the first substrate;
   (b) contacting the biological sample with at least one analyte capture agents, wherein an analyte capture agent comprises a capture agent barcode domain, wherein the capture agent barcode domain comprises an analyte binding moiety barcode and a capture handle sequence;
   (c) aligning the first substrate with a second substrate comprising an array, such that at least a portion of the biological sample is aligned with at least a portion of the array, wherein the array comprises a plurality of capture probes, wherein a capture probe of the plurality of capture probes comprises: (i) a spatial barcode and (ii) a capture domain;
   (d) when the biological sample is aligned with at least a portion of the array, (i) releasing the capture agent barcode domain from the analyte by controlling the temperature of the first substrate in either a heating mode or an off mode; and (ii) passively or actively migrating the capture agent barcode domain from the biological sample to the array; and
   (e) coupling the capture handle sequence to the capture domain.
28. The method of claim 27, further comprising determining (i) all or a part of the sequence of the capture agent barcode domain, or a complement thereof; and (ii) the sequence of the spatial barcode, or a complement thereof.
29. The method of claim 28, further comprising using the determined sequences of (i) and (ii) to determine a location of the analyte in the biological sample.
30. The method of any one of claims 27-29, wherein the determining comprises sequencing (i) all or a part of the capture agent barcode domain, or a complement thereof, and (ii) the spatial barcode, or a complement thereof.
31. A method for analyzing a nucleic acid analyte in a biological sample mounted on a first substrate, the method comprising:
   (g) controlling a temperature of the first substrate in a cooling mode to maintain a fixed state of the biological sample on the first substrate;
   (h) contacting the biological sample with a first probe and a second probe, wherein the first probe and second probe hybridize to the nucleic acid analyte, wherein the first probe and the second probe each comprise a sequence that is substantially complementary to a first and second sequence of the nucleic acid analyte, and wherein the second probe oligonucleotide comprises a capture probe binding domain;
   (i) coupling the first probe and the second probe, thereby generating a connected probe;
   (j) aligning the first substrate with a second substrate comprising an array, such that at least a portion of the biological sample is aligned with at least a portion of the array, wherein the array comprises a plurality of capture probes, wherein a capture probe of the plurality of capture probes comprises: (i) a spatial barcode and (ii) a capture domain;
   (k) when the biological sample is aligned with at least a portion of the array, (i) releasing the connected probe from the nucleic acid analyte by controlling the temperature of the first substrate in either a heating mode or an off mode; and (ii) passively or actively migrating the connected probe from the biological sample to the array; and
   (l) hybridizing the connected probe to the capture domain via the capture probe binding domain.
32. The method of claim 31, wherein the first and second sequence of the nucleic acid analyte abut one another or are at least 1, 2, 3, 4, 5, 6, 7, 8, 9, 10, or more nucleotides away from one another.
33. The method of any one of claims 27-32, wherein the releasing step (d) or step (e) comprises contacting the biological sample and the array with a reagent medium comprising a nuclease, a permeabilization agent, or a combination thereof.
34. The method of any one of claims 27-30, wherein the analyte comprises a protein or a fragment thereof, or a peptide.
35. The method of any one of claims 27-34, wherein the aligning comprises:
   (i) mounting the first substrate on a first member of a support device, the first member configured to retain the first substrate;
   (ii) mounting the second substrate on a second member of the support device,
   (iii) applying the reagent medium to the first substrate and/or the second substrate, and
   (iv) operating an alignment mechanism of the support device to move the first member and/or the second member such that at least a portion of the biological sample is aligned with at least a portion of the array, and such that the portion of the biological sample and the portion of the array contact the reagent medium.
36. The method of claim 35, wherein the alignment mechanism is coupled to the first member, the second member, or both the first member and the second member.
37. The method of claim 35 or 36, wherein the alignment mechanism comprises a linear actuator, optionally wherein:
   the linear actuator is configured to move the second member along an axis orthogonal to a plane or of the first member and/or the second member, and/or
   the linear actuator is configured to move the first member along an axis orthogonal to the plane of the first member and/or the second member, and/or
   the linear actuator is configured to move the first member, the second member, or both the first member and the second member at a velocity of at least 0.1 mm/sec, and/or
   the linear actuator is configured to move the first member, the second member, or both the first member and the second member with an amount of force of at least 0.1 lbs.
38. The method of any one of claims 27-37, wherein during the releasing in step (d) or step (e), a separation distance is maintained between the first substrate and the second substrate, optionally wherein the separation distance is less than 50 microns, optionally wherein the separation distance is between 2-25 microns, optionally wherein the separation distance is measured in a direction orthogonal to a surface of the first substrate upon which the biological sample is mounted, and/or at least the portion of the biological sample is vertically aligned with at least a portion of the array.
39. The method of any one of claims 27-36, wherein at least one of the first substrate and the second substrate further comprise a spacer, wherein after the first and second substrate being mounted on the support device, the spacer is disposed between the first substrate and second substrate and is configured to maintain the reagent medium within a chamber formed by the first substrate, the second substrate, and the spacer, and maintain a separation distance between the first substrate and the second substrate, the spacer positioned to at least partially surround an area on the first substrate on which the biological sample is disposed and/or the array disposed on the second substrate, wherein the area of the first substrate, the spacer, and the second substrate at least partially encloses a volume comprising the biological sample.
40. The method of claim 39, wherein:
   the chamber comprises a partially or fully sealed chamber, and/or
   the second substrate comprises the spacer, and/or
   the first substrate comprises the spacer, and/or
   the applying the reagent medium to the first substrate and/or the second substrate comprises applying the reagent medium to a region of the spacer, the region outside an enclosed area of the second substrate, the enclosed area formed by the spacer.
41. The method of any one of claims 35-40, wherein as the first substrate and/or the second substrate are moved via the alignment mechanism, the first substrate is at an angle relative to the second substrate such that a dropped slide of the first substrate and a portion of the second substrate contact the reagent medium,
   optionally wherein:
      the dropped side of the first substrate urges the reagent medium toward the opposite direction, and/or
      the alignment mechanism further moves the first substrate and/or the second substrate to maintain an approximately parallel arrangement of the first substrate and the second substrate and a separation distance between the first substrate and the second substrate, optionally when the approximately parallel arrangement and the separation distance are maintained, the spacer fully encloses and surrounds the at least portion of the biological sample and the at least portion of the array, and the spacer forms the sides of the chamber which hold a volume of the reagent medium.
42. The method of any one of claims 27-41, wherein the coupling of the capture handle sequence to the capture domain comprises hybridization.
43. The method of any one of claims 33-42, wherein the nuclease comprises an RNase.
44. The method of claim 41, wherein the RNase is selected from RNase A, RNase C, RNase H, and RNase I.
45. The method of any one of claims 33-44, wherein the reagent medium comprises a permeabilization agent, optionally wherein the permeabilization agent comprises a protease, optionally wherein the protease is selected from trypsin, pepsin, elastase, or Proteinase K.
46. The method of any one of claims 27-45, wherein the releasing step further comprises simultaneously permeabilizing the biological sample and releasing the capture agent barcode domain from the analyte binding moiety, or simultaneously permeabilizing the biological sample and releasing the connected probe from the nucleic acid analyte.
47. The method of any one of claim 33-46, wherein the reagent medium further comprises a detergent.
48. The method of claim 47, wherein the detergent is selected from sodium dodecyl sulfate (SDS), sarkosyl, saponin, polyethylene glycol tert-octylphenyl ether, or polysorbate 20.
49. The method of any one of claims 33-48, wherein the reagent medium comprises less than 15 w/v % of a detergent selected from SDS and sarkosyl, or wherein the reagent medium comprises as least 5% w/v % of a detergent selected from SDS and sarkosyl.
50. The method of any one of claims 33-49, wherein the reagent medium does not comprise a detergent and/or a permeabilization agent, preferably wherein the reagent medium does not comprise sodium dodecyl sulfate (SDS) or sarkosyl.
51. The method of any one of claims 33-50, wherein the reagent medium further comprises one or more crowding agents, optionally PEG.
52. The method of any one of claims 33-51, wherein the coupling the first probe and the second probe comprises ligating the first probe and the second.
53. The method of claim 52, wherein the ligating comprises use of a ligase, optionally wherein the ligase is selected from a T4 RNA ligase (Rnl2), a PBCV-1 DNA Ligase or Chlorella virus DNA Ligase, a single-stranded DNA ligase, and a T4 DNA ligase.
54. The method of any one of claims 27-53, wherein the capture probe comprises a poly(T) sequence.
55. The method of any one of claims 27-54, wherein the capture probe comprises a sequence complementary to the capture handle sequence or the capture probe binding domain.
56. The method of any one of claims 27-55, wherein the capture probe further comprises one or more functional domains, a unique molecular identifier (UMI), a cleavage domain, or a combination thereof.
57. The method of any one of claims 27-56, wherein the analyte binding moiety comprises an antibody.
58. The method of any one of claims 27-57, wherein the analyte capture agent comprises a linker, optionally wherein the linker is a cleavable linker.
59. The method of claim 58, wherein the cleavable linker is a photo-cleavable linker, a UV-cleavable linker, or an enzyme cleavable linker.
60. The method of any one of claims 31-59, wherein the nucleic acid analyte is RNA or DNA, preferably wherein the RNA is mRNA.
61. The methods of any one of claims 21-60, wherein the biological sample is a tissue sample.
62. The method of claim 61, wherein the tissue sample is a solid tissue sample.
63. The method of claim 62, wherein the solid tissue sample is a tissue section.
64. The method of claim 62, wherein the tissue section is a fixed tissue section.
65. The method of claim 64, wherein the fixed tissue section is a formalin fixed paraffin embedded (FFPE) tissue section.
66. The method of claim 65, wherein the FFPE tissue is deparaffinized and decrosslinked prior to step (a).
67. The method of claim 63, wherein the tissue section is a fresh frozen tissue section.
68. The method of claim 64, wherein the tissue section is fixed and stained prior to step (a).
69. The method of claim 21, wherein the thermal element is mounted between the upper housing and the substrate.
70. The method of claim 21, wherein the thermal element is mounted to the upper housing and surrounds the substrate.
71. The method of claim 21, wherein the thermal element is a thermoelectric cooler (TEC).
72. The method of claim 21, wherein the thermal element comprises liquid nitrogen.
73. The method of claim 21, wherein the thermal element comprises a cooling liquid that circulates within the thermal element to cool the substrate.
74. The method of claim 27 or 31, wherein the thermal element is mounted between the upper housing and the first substrate.
75. The method of claim 27 or 31, wherein the thermal element is mounted to the upper housing and surrounds the first substrate.
76. The method of claim 27 or 31, wherein the thermal element is a thermoelectric cooler (TEC).
77. The method of claim 27 or 31, wherein the thermal element comprises liquid nitrogen.
78. The method of claim 27 or 31, wherein the thermal element comprises a cooling liquid that circulates within the thermal element to cool the first substrate.
79. A system or kit for analyzing an analyte in a biological sample, the system or the kit comprising:
   (a) a support device configured to retain a first substrate and a second substrate, wherein a biological sample is placed on the first substrate, and wherein the second substrate comprises an array comprising a plurality of capture probes, wherein a capture probe of the plurality of capture probes comprises: (i) a spatial barcode and (ii) a capture domain;
   (b)
      (b1) a first probe and a second probe, wherein the first probe and the second probe each comprise a sequence that is substantially complementary to adjacent sequences of the analyte, wherein the second probe comprises a capture probe binding domain, and wherein the first probe and the second probe e are capable of being ligated together to form a connected probe;
      or
      (b2) a plurality of analyte capture agents, wherein an analyte capture agent of the plurality of analyte capture agents comprises an analyte binding moiety and a capture agent barcode domain, wherein the analyte binding moiety specifically binds to the analyte in the biological sample, and wherein the capture agent barcode domain comprises an analyte binding moiety barcode and a capture handle sequence;
   (c) a reagent medium comprising an agent for releasing the connected probe and optionally a permeabilization reagent; and
   (d) instructions for performing the method of any one of claims 21-68.
80. The system or kit of claim 79, wherein the permeabilization agent is pepsin or proteinase K.
81. The system or kit of claim 79 or 80, wherein the agent for releasing the connected probe is an RNAse, optionally wherein the RNAse is selected from RNase A, RNase C, RNase H, or RNase I.
82. The system or kit of any one of claims 79-81, further comprising an alignment mechanism on the support device to align the first substrate and the second substrate.
83. The system or kit of claim 82, wherein the alignment mechanism comprises a linear actuator, wherein the first substrate comprises a first member and the second substrate comprises a second member, and optionally wherein:
   the linear actuator is configured to move the second member along an axis orthogonal to a plane or of the first member and/or the second member, and/or
   the linear actuator is configured to move the first member along an axis orthogonal to the plane of the first member and/or the second member, and/or
   the linear actuator is configured to move the first member, the second member, or both the first member and the second member at a velocity of at least 0.1 mm/sec, and/or
   the linear actuator is configured to move the first member, the second member, or both the first member and the second member with an amount of force of at least 0.1 lbs.
84. A system comprising:
   an upper housing configured to receive a sample substrate and a thermal element;
   a lower housing; and
   an alignment mechanism coupling the upper housing to the lower housing, the alignment mechanism being configured to move the upper housing, the lower housing, or both into an arrangement having an open configuration, a closed configuration, or an intermediate configuration therebetween;
   wherein the thermal element is configured to cool the sample substrate below room temperature.
85. The system of claim 84, wherein the thermal element is configured to cool the sample substrate after insertion of the sample substrate into the upper housing.
86. The system of claim 84 or 85, wherein the sample substrate comprises a sample window.
87. The system of claim 86, wherein the upper housing further comprises a light emitting diode (LED) assembly, wherein the LED assembly is configured to emit light through the sample window of the sample substrate.
88. The system of any one of claims 84-87, wherein the thermal element comprises a cooling plate.
89. The system of claim 88, wherein the LED assembly is configured to emit light through the thermal element.
90. A method comprising:
   (a) cooling, by a thermal element, a substrate comprising a biological sample such that the biological sample is at or below a threshold temperature;
   (b) connecting a substrate to an upper housing;
   (c) aligning the upper housing with a lower housing by an alignment mechanism, the alignment mechanism being configured to move the upper housing, the lower housing, or both into an arrangement having an open configuration, a closed configuration, or an intermediate configuration therebetween; and
   (d) moving the upper housing towards the lower housing;
   wherein the thermal element is configured to cool the substrate.
91. The method of claim 90, further comprising (b1) connecting the thermal element to the upper housing.
92. The method of claims 90 or 91, wherein the thermal element comprises a cooling plate.
93. A system comprising:
   a thermal element;
   an upper housing configured to receive a sample substrate;
   a lower housing; and
   an alignment mechanism coupling the upper housing to the lower housing, the alignment mechanism being configured to move the upper housing, the lower housing, or both into an arrangement having an open configuration, a closed configuration, or an intermediate configuration therebetween;
   wherein, the thermal element is configured to cool the sample substrate before insertion of the sample substrate into the upper housing.
94. The system of claim 93, wherein the thermal element comprises a cooling plate.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations can be within the scope of the following claims.

What is claimed is:

1. A method comprising:
(a) connecting a first substrate to an upper housing, the first substrate comprising a biological sample;
(b) cooling, by a thermal element of the upper housing, the first substrate such that the biological sample is at or below a threshold temperature;
(c) aligning the upper housing with a lower housing by an alignment mechanism, the alignment mechanism being configured to move the upper housing, the lower housing, or both into an arrangement having an open configuration, a closed configuration, or an intermediate configuration therebetween;
(d) moving the upper housing towards the lower housing;
(e) deactivating the thermal element of the upper housing;
(f) releasing one or more analytes, or proxies thereof, from the biological sample; and
(g) capturing the one or more analytes, or proxies thereof, from the biological sample onto a spatial array disposed on a second substrate, the second substrate mounted on the lower housing.

2. The method of claim 1, wherein step (f) comprises deactivating the thermal element responsive to moving the upper housing and the lower housing from the open configuration into the closed configuration.

3. The method of claim 1, wherein step (d) comprises aligning a base window at a base support member with a sample window at the sample support member.

4. The method of claim 1, wherein step (b) comprises maintaining a surface temperature of the first substrate between −80° C. and 5° C.

5. The method of claim 1, wherein the threshold temperature is in a range of about −5° C. and 5° C., inclusive.

6. The method of claim 1, further comprising: emitting light through a sample window of the first substrate by a light emitting diode (LED) assembly of the upper housing.

7. The method of claim 6, wherein the LED assembly emits light through the thermal element.

8. The method of claim 1, wherein the thermal element is mounted to the upper housing and surrounds the first substrate or is disposed beneath the first substrate.

9. A system or device comprising:
an upper housing configured to receive a sample substrate, the upper housing comprising a thermal element configured to control a temperature of the sample substrate and a light emitting diode (LED) assembly configured to emit light through a sample window of the sample substrate;
a lower housing; and
an alignment mechanism coupling the upper housing to the lower housing, the alignment mechanism being configured to move the upper housing, the lower housing, or both into an arrangement having an open configuration, a closed configuration, or an intermediate configuration therebetween;
wherein the thermal element is configured to cool the sample substrate.

10. The system or device of claim 9, further comprising the sample substrate, wherein the thermal element is mounted between the upper housing and the sample substrate.

11. The system or device of claim 9, wherein the LED assembly is configured to emit light through the thermal element, optionally wherein the thermal element is transparent.

12. The system or device of claim 9, wherein the lower housing further comprises a focus stage, wherein the focus stage comprises a stage, a motor configured to translate the stage, and at least one image sensor positioned on the stage, and at least one lens positioned on the stage.

13. The system or device of claim 9, wherein the thermal element is a thermoelectric cooler (TEC).

14. The system or device of claim 9, wherein in the closed configuration, the thermal element is configured to deactivate.

15. The system or device of claim 9, wherein the lower housing comprises a second thermal element that controls a temperature at a base support member of the lower housing.

16. The system or device of claim 9, wherein the thermal element comprises a cooling liquid that circulates within the thermal element to cool the sample substrate.

17. The system or device of claim 9, wherein the thermal element is configured to, upon energizing the thermal element in a cooling mode, maintain a surface temperature of the sample substrate between −80° C. and 5° C.; and wherein the thermal element is configured to, upon energizing the thermal element in a heating mode, maintain a surface temperature of the sample substrate between 10° C. and 40° C.

18. The system or device of claim 9, wherein the sample substrate comprises a biological sample, wherein the biological sample comprises a tissue section, optionally a fresh frozen tissue section or a fixed tissue section.

19. A method for analyzing a nucleic acid analyte in a biological sample mounted on a first substrate, the method comprising:
(a) controlling a temperature of the first substrate in a cooling mode to maintain a fixed state of the biological sample on the first substrate;
(b) contacting the biological sample with a first probe and a second probe, wherein the first probe and second probe hybridize to the nucleic acid analyte, wherein the first probe and the second probe each comprise a sequence that is substantially complementary to a first and second sequence of the nucleic acid analyte, and wherein the second probe oligonucleotide comprises a capture probe binding domain;
(c) coupling the first probe and the second probe, thereby generating a connected probe;
(d) aligning the first substrate with a second substrate comprising an array, such that at least a portion of the biological sample is aligned with at least a portion of the array, wherein the array comprises a plurality of capture probes, wherein a capture probe of the plurality of capture probes comprises: (i) a spatial barcode and (ii) a capture domain;
(e) when the biological sample is aligned with at least a portion of the array, (i) releasing the connected probe from the nucleic acid analyte by controlling the temperature of the first substrate in either a heating mode or an off mode; and (ii) passively or actively migrating the connected probe from the biological sample to the array; and
(f) hybridizing the connected probe to the capture domain via the capture probe binding domain.

20. A system or kit for analyzing an analyte in a biological sample, the system or the kit comprising:
(a) a support device configured to retain a first substrate and a second substrate, wherein a biological sample is placed on the first substrate, and wherein the second substrate comprises an array comprising a plurality of capture probes, wherein a capture probe of the plurality of capture probes comprises: (i) a spatial barcode and (ii) a capture domain;
(b)
(b1) a first probe and a second probe, wherein the first probe and the second probe each comprise a sequence that is substantially complementary to adjacent sequences of the analyte, wherein the second probe comprises a capture probe binding domain, and wherein the first probe and the second probe e are capable of being ligated together to form a connected probe;
or
(b2) a plurality of analyte capture agents, wherein an analyte capture agent of the plurality of analyte capture agents comprises an analyte binding moiety and a capture agent barcode domain, wherein the analyte binding moiety specifically binds to the analyte in the biological sample, and wherein the capture agent barcode domain comprises an analyte binding moiety barcode and a capture handle sequence;
(c) a reagent medium comprising an agent for releasing the connected probe and optionally a permeabilization reagent; and
(d) instructions for performing the method of claim 1.
